(19) United States
(12) Patent Application Publication
(10) Pub. No.: US 2011/0158998 A1
(43) Pub. Date: Jun. 30, 2011

(54) HELMINTH ANTIGEN AND IMMUNOTHERAPY

(75) Inventors: Alexander Constantine Loukas, Queensland (AU); Mai Tran, Queensland (AU); Malcolm Kenneth Jones, Queensland (AU); Danielle Julianna Smyth, Queensland (AU); Donald Peter Mcmanus, Queensland (AU); Mark Simon Pearson, Queensland (AU); Jeffrey Michael Bethony, Queensland (BR)

(73) Assignee: THE COUNCIL OF THE QUEENSLAND INSTITUTE OF MEDICAL RESEARCH, Queensland (AU)

(21) Appl. No.: 11/997,714

(22) PCT Filed: Jun. 9, 2006
(86) PCT No.: PCT/AU2006/000807

§ 371 Date: Feb. 11, 2011

(30) Foreign Application Priority Data
Aug. 3, 2005 (AU) 2005904161

(51) Int. Cl. A61K 39/395; C07K 14/00; C07K 16/00; A61K 39/00; C12N 15/31; C12N 1/00; C12P 21/00; G01N 33/53; A61P 37/04; A61P 33/12

(52) U.S. Cl. 424/139.1; 530/350; 530/324; 530/389.1; 424/191.1; 536/23.7; 435/243; 435/69.1; 436/501

(57) ABSTRACT

A helminth protein immunogen is described. Included as the immunogen is an isolated protein comprising an immunogenic, extracellular fragment of a schistosome tegument protein selected from the group consisting of a TSP-1 protein; a TSP-2 protein; and a 7TM protein. An antibody which binds the isolated protein is also described. Additionally, an immunotherapeutic composition comprising the immunogenic protein and an immunologically acceptable carrier, diluent or excipient is described. Furthermore, a vaccine comprising the immunogenic protein is described. Also described are an isolated nucleic acid that encodes the immunogenic protein and a genetic construct comprising that isolated nucleic acid. Further, a host cell comprising the genetic construct is described. A method of immunizing against schistosomiasis including the step of administering the immunotherapeutic composition to an animal is also described. Other methods described include prophylactic or therapeutic treatment of schistosomiasis and of determining whether an animal has been exposed to, or harbours, a schistosome.

### FIELD OF THE INVENTION

THIS INVENTION relates to a helminth protein immunogen. More particularly, this invention relates to an extracellular domain fragment of a schistosome tegument protein and/or an encoding nucleic acid, for use in immunotherapy and/or diagnosis of schistosomiasis.

### BACKGROUND OF THE INVENTION

Human infections arising from helminths such as Schistosoma mansoni (S. mansoni) signify a considerable portion of the global burden of illness with over 83 million people infected in 54 countries across Africa, the eastern Mediterranean, South America and the Caribbean (Lebens et al., 2004, Expert Rev Vaccines 3 315-28).

The two other medically important schistosome species—S. japonicum and S. haematobium—exacerbate this burden, infecting 2 million and 113 million individuals, respectively (Crompton, 1999, J Parasitol 85 397-403).

Collectively, the at-risk population numbers over 600 million, with over 85% of these individuals inhabiting sub-Saharan Africa (Utzinger et al., 2003, Lancet 362 1932-34).

Second only to malaria in terms of severe parasite-induced morbidity, it is estimated that 20 million people suffer from severe consequences of this chronic and debilitating disease, responsible for hundreds of thousands of deaths each year (Chitsulo et al., 2000, Acta Trop 77 44-51; van der Werf et al., 2003, Acta Trop 86 125-139).

Recent meta-analysis and recalculations of DALY (disability adjusted life year) estimates have shown that the burden of schistosomiasis impacts far more substantially than previously appreciated. Further, the effect of schistosomiasis on other disease interventions and promotion of nutrition and public health in general has been underestimated (Capron et al., 2005, Trends Parasitol. 21 143-149).

The perpetuation of the S. mansoni life cycle and, therefore, the incidence of infection, is reliant on two main epidemiological factors: (i) the exposure of humans to fresh water habituated by a compatible snail host and (ii) the contamination of these fresh water habitats with human organic waste (Engels et al., 2002, Acta Trop. 82 139-146). Thus, it is the countries who are dependent on snail infested waterways and dispose of waste into these habitats that create an environment which is amenable to schistosome infection.

Schistosoma mansoni flatworms are digenetic, blood-dwelling flukes belonging to the class Trematoda. Adult worms of S. mansoni are dioecious and typically 7-20 mm in length, with males being shorter and stouter than the females. Both sexes possess two suckers: a weak oral sucker perforated by the mouth and a more muscular acetabulum near the anterior end (Roberts and Janovy, 2000. In Foundations of Parasitology. Wm. C. Brown, Chicago, pp. 233-250)

Like other platyhelminths, the entire body is covered with a tegument, which is a continuous, metabolically active, acellular layer bound externally by a plasma membrane (Sturrock, 2001. In: Mahmoud A. F. (Ed.), Schistosomiasis. Imperial College Press, London, pp. 7-83). Cercariae of S. mansoni are approximately 0.5 mm in length and have a bifurcated tail. Like adult worms, S. mansoni cercariae possess both oral and ventral suckers and a tegumental surface; the cercarial tegument, however, is additionally covered by a glycocalyx and only encapsulates the body and not the tail (Sturrock, 2001, supra).

S. mansoni is an unusual trematode in that it uses only one intermediate host—freshwater snails of the genus Biomphalaria—for the completion of its life cycle. Humans are the predominant definitive hosts in the S. mansoni life cycle, in which adult development and egg production occurs (Roberts and Janovy, 2000, supra; Sturrock, 2001, supra).

Following penetration of unbroken skin of the definitive host, cercariae transform into schistosomula and enter the vasculature to the sinuses of the liver.

Immature flukes develop in the liver and pairing between males and females occur as they reach maturity. Coupled worms migrate through the hepatic portal system to the veins around the bladder where eggs are laid. The eggs then enter the intestinal lumen where they are voided in the faeces and, upon reaching fresh water, hatch and release free living miracidia. Following infiltration of the intermediate snail host, the miracidia develop into cercariae, which are subsequently shed by the snail. Completion of the life cycle occurs when cercariae penetrate the skin of a definitive host (Roberts and Janovy, 2000, supra; Sturrock, 2001, supra).

Infection with S. mansoni causes intestinal schistosomiasis, a disease which results in the formation of damaging inflammatory and fibrotic lesions in the infected human host in response to the presence of eggs in the intestinal veins and other parts of the body. An initial, immune complex-mediated reaction in response to egg antigens (acute schistosomiasis) occurs up to 3 months after cercarial penetration with common symptoms including fever, headache, generalised myalgia and bloody diarrhoea (Ross et al., 2002, N. Engl. J. Med. 346 1212-20).

Chronic schistosomiasis occurs if the infection progresses unchecked or without treatment. It is characterised by an inflammatory, granulomatous response at the site of egg deposition which ultimately destroys the ova but results in fibrous plaques in the host tissue. As the principal sites of egg deposition are the gut and liver and the associated vasculature, granuloma formation in these areas can induce inflammation, fibrosis and ulceration. Further, eggs retained in the intestinal lumen can cause diarrhoea and polyposis while ova deposited in the liver often results in hepatomegaly and periportal fibrosis, which can lead to hypertension and the obstruction of blood flow. Eggs that fail to migrate to the intestinal lumen or liver can be deposited, via the circulatory system, in other tissues including the skin, lung, brain, adrenal glands and skeletal muscle. Inflammatory masses in these areas can accumulate (often undetected) over time, resulting in a disabling, sometimes fatal, disease. Furthermore, at-risk individuals with diseases such as hepatitis B and C and liver cirrhosis are a concern as co-infection with schistosomiasis can lead to the exacerbation of hepatopathology associated with these conditions (Ross et al., 2002, supra).

The boundary that forms the interface between S. mansoni and its host environment is represented by the tegument. Furthermore, this structure is integral in mediating the cross-talk between the opposing sides, relaying messages that are pivotal to the establishment of infection.

The tegument of S. mansoni comprises a cytoplasmic syncitium enclosed by an outer membrane. Internally, a basal laminar membrane, consisting of a single lipid bilayer (McLaren and Hockley, 1977, Nature 269 147-149), which extends beneath the granular, tegumental cytoplasm and covers a thick layer of muscle fibres and subtegumental cells (Hockley and McLaren, 1973, Int. J. Parasitol. 3 13-25; Jones et al., 2004, Bioessays 26 752-765).

While the basic architecture of the tegument remains the same throughout the schistosome life cycle, other tegumental transformations occur during each developmental stage. In cercariae, the tegumental outer membrane consists of a single lipid bilayer and is covered by a layer of diffuse and granular material—the glycocalyx—and a sparse distribution of mitochondria along with more numerous spheric bodies can be seen in the cytoplasm (Hockley, 1972, Parasitology 64 245-252).

After skin penetration, the transformation of cercariae into schistosomula is predominantly characterized by the replacement of the outer membrane with a double lipid bilayer, which is formed from numerous membranous vacuoles that pass from the subtegumental cells, via a network of connecting tubules (Morris and Threadgold, 1968, Journal of Parasitology 54 15-27), and open up onto the tegumental membrane. This is accompanied by the casting off of the original membrane and the shedding of the glycocalyx (Hockley and McLaren, 1973, supra). The tegumental architecture of the adult worm is generally similar to the schistosomula, with the exception of a comparatively invaginated tegumental surface, less dense cytoplasmic material and a greater number of membranous inclusions, the presence of which are implicated in the continuous breakdown and reformation of the outer membrane (Hockley and McLaren, 1973, supra; Jones et al., 2004, supra).

The double lipid bilayered tegumental membrane, while an unusual structure, is a feature shared by all of the schistosomatidae and is also present in members of the other blood fluke families, the aporocotylidae and the spirorchiidae (McLaren and Hockley, 1977, supra). Contrastingly, other flukes which inhabit the gut and associated body cavities have a tegument covered by a single lipid bilayer. The unusual tegumental structure of the blood flukes, therefore, would seem to be an adaptation to survival in the vertebrate blood stream (Abath and Werkhauser, 1996, Parasite Immunol. 18 15-20).

In addition to its structural properties, the schistosome tegument plays an active role in nutrition, assimilating simple host nutrients such as amino and fatty acids, simple sugars, inorganic ions and lipids (Sturrock, 2001, supra) in an active, energy-dependent fashion (Ribeiro et al., 1998, Parasitology 116 229-236). In paired adults, the positioning of the female prevents her from host serum contact and so nutrients are transported from the male to the female through their teguments, which are intimately associated with one another (Sturrock, 2001, supra).

The tegument also constitutes the primary line of defense against the host immune system and, as such, displays an array of striking immunoevasive strategies (Sturrock, 2001, supra; Maizels et al., 2004, Immunol. Rev. 201 89-116). Such mechanisms include: neutralization of host clotting mechanisms (Tsang and Damian, 1977, Blood 49 619-633) and toxic oxidants (Mei and LoVerde, 1997, Exp. Parasitol. 86 69-78); acquisition of host molecules onto the tegument to mask parasite antigens (Clegg et al., 1971, Nature 232 653-4 ; Loukas et al., 2001, Infect. Immun. 69 3646-3651); removal of tegument-associated immune complexes by continual membrane shedding (Pearce et al., 1986, Parasite Immunol. 8 79-89); and the inactivation of immunomodulatory molecules by proteases (Fishelson, 1995, Mem. Inst. Oswaldo. Cruz. 90 289-292).

The reproducible induction of protective immunity in mice elicited by irradiated cercariae and antigenic preparations has provided evidence that an anti-schistosome vaccine is a possible goal (Bergquist et al., 2002, Acta Trop 82 183-192). However, fewer than 10% of vaccine candidates discovered have progressed further than initial animal trials due to generally disappointing protective efficacies in these models and, where tested, poor performance in human correlate studies (Capron et al., 2005, supra).

### SUMMARY OF THE INVENTION

The invention is therefore broadly directed to an isolated protein which comprises at least an immunogenic fragment of a schistosome tegument protein, or a tegument protein of other helmiths such as liver flukes, which is capable of eliciting an immune response upon administration to an animal.

In particular forms, schistosomes include, but are not limited to, S. mansoni, S. japonicum and/or S. haematobium.

A preferred example of a liverfluke is Fasciola hepatica.

In a first aspect, the invention provides an isolated protein comprising an immunogenic, extracellular fragment of a schistosome tegument protein selected from the group consisting of a TSP-1 protein; a TSP-2 protein; and a 7TM protein.

Suitably the isolated protein is not a full-length schistosome tegument protein.

In one embodiment, the isolated protein may consist of or consist essentially of, an immunogenic, extracellular fragment of a schistosome tegument protein selected from the group consisting of a TSP-1 protein; a TSP-2 protein; and a 7TM protein.

Preferably, the schistosome tegument protein is a tetraspanin protein.

In particular embodiments, the schistosomal tetraspanin protein is a TSP-1 or a TSP-2 protein.

In a preferred embodiment, the schistosomal tetraspannin protein is a TSP-2 protein.

In a particularly preferred embodiment, the immunogenic, extracellular fragment is amino acids 107-184 of S. mansoni TSP-2.

In a second aspect, the invention provides an antibody which binds the immunogenic, extracellular fragment of the isolated protein of the first aspect.

In a third aspect, the invention provides an immunotherapeutic composition comprising one or more immunogenic proteins of the first aspect and/or the antibody of the second aspect together with an immunologically acceptable carrier, diluent or excipient.

In a particular embodiment, the immunotherapeutic composition comprises an immunogenic extracellular fragment of a TSP-2 protein, alone or together with a TSP-1 protein fragment and/or a 7TM protein fragment.

In another particular embodiment, the immunotherapeutic composition is a vaccine.

In a fourth aspect, the invention provides an isolated nucleic acid that encodes the immunogenic protein of the first aspect.

In a particular embodiment the isolated nucleic acid encodes amino acids 107-184 of a S. mansoni TSP-2 amino acid sequence set forth in SEQ ID NO:5.

In another particular embodiment the isolated nucleic acid comprises nucleotides 321-554 of the S. mansoni TSP-2 nucleotide sequence set forth in SEQ ID NO:2.

In a fifth aspect, the invention provides a genetic construct comprising the isolated nucleic acid of the fourth aspect operably linked or connected to one or more regulatory nucleotide sequences.

In a sixth aspect, the invention provides a host cell comprising the genetic construct of the fifth aspect.

In a seventh aspect, the invention provides an immunotherapeutic composition comprising the genetic construct of the fifth aspect together with an immunologically acceptable carrier, diluent or excipient.

In an eighth aspect, the invention provides a method of immunizing against schistosomiasis, or a related disease or condition, including the step of administering the immunotherapeutic composition of the third aspect or the seventh aspect to an animal.

In a ninth aspect, the invention provides a method of prophylactic or therapeutic treatment of schistosomiasis, or a related disease or condition, including the step of administering the immunotherapeutic composition of the third aspect or the seventh aspect to an animal.

According to the aforementioned aspects the animal may be a mammal, preferably a human.

In a tenth aspect, the invention provides a method of producing a recombinant protein including the step of expressing the isolated nucleic acid of the fourth aspect in one or more host cells to thereby produce said isolated protein.

Preferably, the method includes the further step of purifying the isolated protein from the host cell(s).

In an eleventh aspect, the invention provides a method of determining whether an animal has been exposed to, or harbours, a schistosome, said method including the step of determining whether a biological sample obtained from said animal comprises one or more antibodies which bind an immunogenic, extracellular fragment of a schistosome tegument protein selected from the group consisting of a TSP-1 protein; a TSP-2 protein; and a 7TM protein, wherein a presence of said one or more antibodies indicates that said animal has been exposed to, or harbours, a schistosome.

Preferably, the biological sample is serum.

Preferably, the animal is a human.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE DRAWINGS

FIG. 1. Nucleic acid sequence of Sm-TSP-1, referred to herein as SEQ ID NO: 1.

FIG. 2. Nucleic acid sequence of Sm-TSP-2, referred to herein as SEQ ID NO: 2.

FIG. 3. Nucleic acid sequence of Sm7-TM, referred to herein as SEQ ID NO: 3.

FIG. 4. Amino acid sequence of Sm-TSP-1, referred to herein as SEQ ID NO: 4.

FIG. 5. Amino acid sequence of Sm-TSP-2, referred to herein as SEQ ID NO: 5.

FIG. 6. Amino acid sequence of Sm7-TM, referred to herein as SEQ ID NO: 6.

FIG. 7. Flowchart showing mouse vaccination and subsequent parasite challenge schedule. Four groups of 10 female CBA/CaH mice were used: 3 groups each being vaccinated with a different test antigen and 1 control group being vaccinated with PBS. FCA=Freund's complete adjuvant; FIA=Freund's incomplete adjuvant.

FIG. 8. Expression of Sm-TSP-1-EC2. (A) SDS-polyacrylamide gel stained with Coomassie Brilliant Blue showing soluble protein profiles from E. coli before (lane 1) and 4 hours after (lane 2) induction with 0.2% arabinose. Sm-TSP-1-EC2 was subsequently purified by metal affinity resin and analysed by (B) SDS-PAGE and (C) Western blotting using anti-6× His-HRP. Molecular weight markers are shown in kDa.

FIG. 9. Expression of Sm-TSP-2-EC2. (A) SDS-polyacrylamide gel stained with Coomassie Brilliant Blue showing soluble protein profiles from E. coli before (lane 1) and 4 hours after (lane 2) induction with 0.2% arabinose. Sin-TSP-2-EC2 was subsequently purified by metal affinity resin and analysed by (B) SDS-PAGE and (C) Western blotting using anti-6× His-HRP. Molecular weight markers are shown in kDa.

FIG. 10. Western blot showing vaccine antigen detection by its corresponding antisera. (A) Blot contains Sm-7TMC. (B) Blot contains Sin-TSP-1-EC2. (C) Blot contains Sm-TSP-2-EC2. Lane 1—antigen-specific antiserum. Lane 2—pre-immune serum. Molecular weight markers are shown in kDa.

FIG. 11. Serum antibody titres in response to immunisation and antigen recognition of experimentally infected mouse sera. (A) Sm-TSP-2-EC2. (B) Sm-TSP-1-EC2. (C) Sm-7TMC. P.I.I=post initial immunisation.

FIG. 12. Detection of native proteins in adult Schistosoma mansoni extracts. (A) Blot contains proteins precipitated from a biotinylated, IgG-cleared, TritonX-100-solubilised extract with anti-Sin-TSP-2-EC2 serum. Lanes 1 and 2 contain unbound and bound fractions, respectively, after precipitation with pre-immune serum. Lanes 3 and 4 contain unbound and bound fractions, respectively, after precipitation with anti-Sm-TSP-2-EC2 serum. The arrows denote the native Sm-TSP-2 molecule and its putative binding partner.

FIG. 13. Comparison of mean worm and egg burdens and liver size between immunised and control animals. (A) Total worm burden. (B) Adult worm burden. (C) Female worm burden. (D) Liver egg burden. (E) Liver weight.

FIG. 14. Antigen recognition by experimentally infected mouse and naturally infected human sera. (A) Schistosoma mansoni SDS-soluble extract. (B) Sm-TSP-2-EC2. (C) Sm-TSP-1-EC2. (D) Sin-7TMC. (E) E.coli thioredoxin. EN =endemic normal. CI=chronically infected.

FIG. 15. Immunofluorescence of methanol fixed adult S. mansoni sections with anti-Sm-TSP-1, anti-Sm-TSP-2 and anti-thioredoxin sera followed by Cy2-conjugated antimouse Ig. Magnifications are denoted in parentheses. Fluorescence denotes regions where antibody has bound. Sections were also stained with DAPI to label nuclei (blue). teg—tegument; tub—tubercles of the tegument; nuc-nuclei. Note fluorescence of just the tegument with antibodies to TSP-1 and TSP-2 but not antibodies to thioredoxin. Some of the nuclei immediately beneath the tegument (arrowed) are probably from tegumentary cytons (see Jones et al., 2004 Bioessays 26 752-65) and likely represent the site of synthesis for tegument proteins before they are trafficked to the tegument syncitium—some of these putative tegumentary cyton nuclei are associated with green fluorescence.

FIG. 16. Individuals who are putatively resistant to S. mansoni selectively recognize Sm-TSP-2 but chronically infected individuals do not. Serum IgG1 and IgG3 response to Sm-TSP-2 from: individuals exposed to S. mansoni but uninfected (PR); individuals exposed and chronically infected with different infection intensities (Light=1-99 epg; Medium=100-399 epg; Heavy=400+epg); unexposed blood donors from the U.S.A. (USA Neg) and unexposed blood donors from Brazil (BRAZ Neg) are shown. Antibody responses were determined by ELISA. Dots reflect OD values of individual patients and bars reflect the mean OD values of the groups. IgG2, IgG4 and IgE responses were assessed but not detected in any of the groups (not shown).

### DETAILED DESCRIPTION OF THE PREFERRED EMBODIMENT

The present invention relates to, at least in part, an isolated, immunogenic protein that comprises at least a fragment of an extracellular domain of one or more schistosome tegument protein, or a tegument protein of other helmiths such as liver flukes, which is capable of eliciting an immune response upon administration to an animal.

In particular forms the helminth may be a trematode. Particular trematodes include a schistosome or flukes, such as, liver flukes. Examples of schistosomes include, but are not limited to, S. mansoni, S. japonicum and/or S. haematobium. An example of a liver fluke is Fasciola hepatica.

In another particular form the helminth protein is a tegumental transmembrane protein of a schistosome, selected from the orphan 7TM receptor, 7TM, and two members of the “tetraspanin” family of proteins, TSP-1 and TSP-2.

The immunogenic extracellular domains of these tegument proteins are set forth herein as new and attractive candidates for immunotherapeutic strategies as they are likely to be accessible to the host immune system during the course of infection.

In particular, an immunogenic fragment of Sin-TSP-2, the TSP-2 protein of S. mansoni, has been shown to be particularly efficacious in terms of reducing worm burden and liver egg burden in a murine model of schistosomiasis. It is therefore contemplated that this immunogenic fragment may be useful in immunotherapy either alone or in combination with other immunogens such as immunogenic fragments of Sm-7TM, and/or Sm-TSP-1.

Suitably the isolated, immunogenic protein is not a full-length schistosome tegumental transmembrane protein. Thus the isolated protein encompasses, for example, a chimeric protein that comprises the immunogenic, extracellular fragment of the invention and other heterologous sequences. Suitable examples of heterologous sequences include, but are not limited to, a fusion partner and/or an epitope tag and/or other heterologous amino acid sequences.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

By “protein” is meant an amino acid polymer. The amino acids may be natural or non-natural amino acids, D- or L-amino acids as are well understood in the art. Also included within the scope of amino acids are chemically-modified or derivatized amino acids as are well known in the art.

A “peptide” is a protein having less than fifty (50) amino acids.

A “polypeptide” is a protein having fifty (50) or more amino acids.

According to one aspect of the invention, there is provided an immunogenic protein comprising at least a fragment of an extracellular domain of a schistosome tegument protein selected from the group consisting of: a TSP-1; a TSP-2 protein; and a 7TM protein.

The tegument protein may be of a schistosome of any species of the genus Schistosoma, including but not limited to pathogenic schistosomes such as S. mansoni, S. japonicum and/or S. haematobium.

Amino acid sequences of TSP-1, TSP-2 and 7TM receptor are respectively set forth in SEQ ID NOS:4-6.

By “extracellular fragment” in this context is meant a topographically identifiable region, portion, domain or sub-sequence of a schistosome tegument protein which, in its normally expressed state, is located outside of, or external to, the plasma membrane of a cell expressing the tegument protein.

Preferably, the schistosome tegument protein is a tetraspanin protein.

In particular embodiments, the schistosomal tetraspanin protein is a TSP-1 (SEQ ID NO:4) or a TSP-2 protein (SEQ ID NO:5).

In a preferred embodiment, the schistosomal tetraspanin protein is a TSP-2 protein.

In a particularly preferred embodiment, the isolated protein comprises amino acids 107-184 of an S. mansoni TSP-2 protein (SEQ ID NO:5), which is the amino acid sequence of extracellular domain 2 (EC2) of the TSP-2 protein.

It will be appreciated that the isolated protein may comprise the immunogenic, extracellular fragment of the tegument protein, together with one or more other amino acids, although the invention specifically excludes the corresponding full length tegument protein.

In one embodiment, the isolated protein may consist essentially of the immunogenic, extracellular fragment of the tegument protein.

By “consist essentially of” is meant that one, two, three, four, five or no more than six additional amino acids are present at the N- and/or C-terminus of the immunogenic, extracellular fragment of the tegument protein.

It will be appreciated, for example, that additional amino acid sequences may be present due to the addition of a fusion partner, as a result of nucleic acid cloning or due to the presence of other immunogenic sequences.

In another embodiment, the isolated protein may consist of the immunogenic, extracellular fragment of the tegument protein to the exclusion of other amino acids.

In the context of the present invention, “immunogenic” describes a capability to elicit an immune response when administered to an animal, preferably a mammal such as a human.

Suitably, the immune response includes at least an antibody response.

Preferably, the immune response is a protective immune response.

It is to be understood the isolated protein may comprise an immunogenic fragment, portion, region or segment, contiguous or non-contiguous, of the extracellular domain of said tegument protein.

In particular embodiments, said immunogenic fragment, portion, region or segment may comprise at least eight (8), nine (9), ten (10), twelve (12), fifteen (15), twenty (20), twenty-five (25), thirty (30), forty (40), fifty (50), sixty (60) or seventy (70) contiguous or non-contiguous amino acids of said extracellular domain of said tegument protein.

Accordingly, the immunogenic fragment, portion, region or segment suitably comprises one or more immunogenic epitopes and is thereby capable of eliciting an immune response upon administration to an animal.

By “epitope” is meant a contiguous or non-contiguous sequence of amino acids that is recognized by at least one T cell or B cell clonotype in vivo or in vitro.

In certain embodiments, isolated proteins of the invention may comprise a plurality of immunogenic fragments as described herein and, optionally, additional amino acid residues.

By way of example, an isolated protein of the invention may comprise multiple copies of the same immunogenic fragment and/or may comprise a plurality of different immunogenic fragments of the same or different schistosome tegument protein and/or immunogenic sequences from other schistosome antigens.

A particular example of this type of isolated protein is a “polyepitope” construct as is well known in the art.

The invention also contemplates variants and/or derivatives or other modified immunogenic fragments that nevertheless retain immunogenicity.

In particular embodiments, isolated proteins of the invention may include one or more amino acid insertions, deletions, substitutions and/or other modifications, as is well understood in the art.

Generally, conservative amino acid substitutions may be introduced to an immunogenic protein or peptide of the invention that essentially retain the immunogenicity. Alternatively, non-conservative amino acid substitutions may be introduced that modify immunogenicity as desired.

Also contemplated are variants that are the result of natural variation in the schistosome genome and encode natural variants of an immunogenic, extracellular domain fragment of a schistosome tegument protein selected from the group consisting of TSP-1 protein; TSP-2 protein; and 7TM protein.

Accordingly, the invention also contemplates immunogenic, extracellular fragments of orthologous TSP-1, TSP-2 and/or 7TM proteins isolated from schistosomes other than S. mansonii.

Examples of homologs of S. mansonii TSP-2 include:

(i) S. japonicum BU803133 which has 75% identity over 209 amino acids and 56% identity over EC-2 region.

(ii) S. japonicum BU798238 which has 77% identity over 217 amino acids and 58% identity over EC-2 region and which is 92% identical to BU803133.

An example of a homolog of TSP-1 is S. japonicum BU793431 which has 97% identity over 201 amino acids.

The invention also contemplates immunogenic, extracellular fragments of homologous and/or orthologous TSP-1, TSP-2 and/or 7TM proteins isolated from other Helminths, particularly trematodes, such as the liver fluke Fasciola hepatica.

In light of the foregoing, in particular embodiments variants of the immunogenic proteins of the invention may comprise an amino acid sequence having at least 55%, preferably at least 60%, more preferably at least 65% and advantageously at least 70%, 75%, 80%, 81%, 82%, 83%, 84%, 85%, 86%, 87% 88%, 89%, 90%, 91%, 92%, 93%, 94%, 95%, 96%, 97%, 98% or 99% sequence identity with an amino acid sequence of an immunogenic extracellular fragment of a schistosome tegument protein.

Sequence identity may be measured over a “comparison window” of at least six amino acids, preferably at least twelve amino acids, more preferably at least twenty amino acids and advantageously over substantially the entire length of a reference amino acid sequence.

It is also contemplated that isolated proteins of the invention may be chemically cross-linked to a carrier protein (such as BSA or thyroglobulin). Other types of chemical modifications of amino acids are well known in the art, although the skilled person is referred to Chapter 15 of CURRENT PROTOCOLS IN PROTEIN SCIENCE Eds. Coligan et al. John Wiley & Sons NY USA (1995-2001) for more detailed methodology relating to chemical modification of proteins.

By way of example only, derivatives contemplated by the invention include, but are not limited to, modification to side chains, incorporation of non-natural amino acids and/or their derivatives during protein synthesis and the use of crosslinkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; and carboxymethylation with iodoacetic acid or iodoacetamide.

Examples of incorporating non-natural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

Isolated proteins, immunogenic peptides derived therefrom, variants and the like may be produced by recombinant DNA technology or by solid or liquid phase chemical synthesis as are well known in the art.

Recombinant protein expression is well known in the art and expression systems are available in bacterial (e.g E. coli DHSα), insect (e.g. Sf9), yeast and mammalian cells.

By way of example, the skilled person is referred to Chapters 5 and 18 of CURRENT PROTOCOLS IN PROTEIN SCIENCE Eds. Coligan et al. John Wiley & Sons NY USA (1995-2001) for techniques applicable to recombinant protein expression and chemical synthesis respectively.

Alternatively, peptides can be produced by digestion of an isolated protein with proteinases such as endoLys-C, endoArg-C, endoGlu-C and staphylococcus V8-protease. The digested fragments can be purified by, for example, high performance liquid chromatographic (HPLC) techniques.

For the purposes of recombinant protein expression, a fusion partner may be added to assist purification. By way of example, fusion partners include a polyhistidine tag, maltose binding protein (MBP), Protein A and glutathione S-transferase (GST) and “epitope tags” such as c-myc, haemagglutinin, V5 and FLAG tags, although without limitation thereto.

It will also be appreciated that protease cleavage sites (e.g. factor Xa and thrombin sites) may be present between the isolated protein and fusion partner to allow subsequent removal of the fusion partner.

### Isolated Nucleic Acids and Genetic Constructs

In one form, the invention provides an isolated nucleic acid encoding one or more immunogenic proteins of the invention.

In particular embodiments, said isolated nucleic acids encode immunogenic, extracellular fragments of one or more tegumental transmembrane proteins of schistosomes selected from the orphan 7TM receptor, 7TM, and two members of the “tetraspanin” family of proteins, TSP-1 and TSP-2.

Amino acid sequences of TSP-1, TSP-2 and 7TM receptor are respectively set forth in SEQ ID NOS:4-6.

Encoding nucleotide sequences are set forth in SEQ ID NOS:1-3.

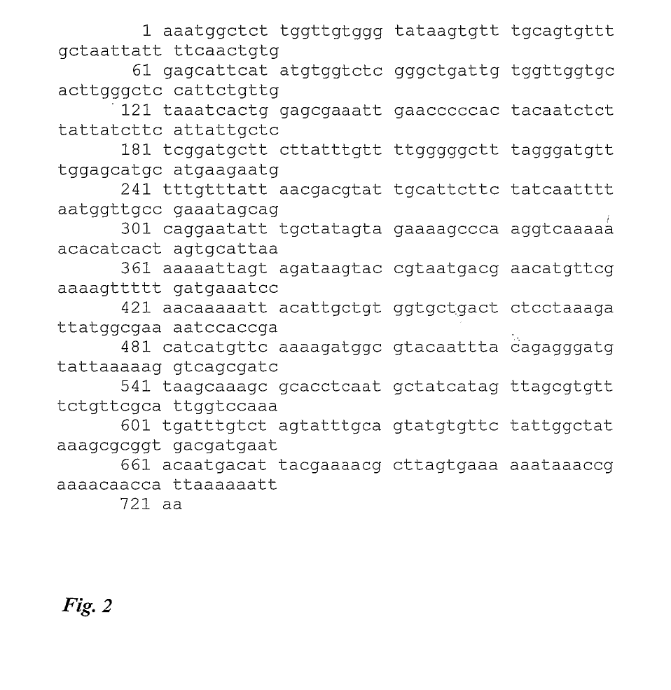

In a particular embodiment said isolated nucleic acid comprises nucleotides 321-554 of S. mansoni TSP-2 shown in SEQ ID NO: 2 and FIG. 2.

Also contemplated are isolated nucleic acids encoding an immunogenic fragment, portion, region or segment, contiguous or non-contiguous, of the extracellular domain of said tegument protein, as hereinbefore described.

The term “nucleic acid” as used herein designates single-or double-stranded mRNA, RNA, cRNA, RNAi and DNA, inclusive of cDNA, genomic DNA and DNA-RNA hybrids.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

The invention also contemplates use of modified purines (for example, inosine, methylinosine and methyladenosine) and modified pyrimidines (thiouridine and methylcytosine) in nucleic acids of the invention.

The present invention also contemplates isolated nucleic acids that have been modified such as by taking advantage of codon sequence redundancy. In a particular example, codon usage may be modified to optimize expression of a nucleic acid in a particular organism or cell type and/or to enhance the immunogenicity of an expressed protein.

Also contemplated are variant nucleic acids that are the result of natural variation in the schistosome genome and encode natural variants of an immunogenic, extracellular domain fragment of a schistosome tegument protein selected from the group consisting of TSP-1 protein; TSP-2 protein; and 7TM protein.

Modified and/or variant nucleic acids may have at least 45%, preferably at least 50%, more preferably at least 60% and advantageously at least 65%, 70%, 75%, 80%, 81%, 82%, 83%, 84%, 85%, 86%, 87% 88%, 89%, 90%, 91%, 92%, 93%, 94%, 95%, 96%, 97%, 98% or 99% nucleotide sequence identity with a nucleotide sequence encoding an immunogenic, extracellular domain fragment of a schistosome tegument protein selected from the group consisting of TSP-1 protein; TSP-2 protein; and 7TM protein.

Sequence identity may be measured over a “comparison window” of at least twelve nucleotides, preferably at least twenty nucleotides, more preferably at least fifty nucleotides and advantageously over substantially the entire length of a reference nucleotide sequence.

It is also contemplated that modified and/or variant nucleic acid of the invention may include one or more insertions, deletions, substitutions and/or other modifications.

The invention also provides a genetic construct comprising an isolated nucleic acid of the invention, operably linked or connected to one or more regulatory nucleotide sequences.

The genetic construct may be adapted for use as follows:
(i) for propagation and/or recombinant protein expression in bacteria;
(ii) for recombinant protein expression in eukaryotic cells (e.g. yeast, Sf9 or mammalian cell lines); and/or
(iii) for delivery to an animal as an immunotherapeutic composition.

By “operably linked” or “operably connected” is meant that an isolated nucleic acid of the invention is under the regulatory control of one or more regulatory sequences which initiate, maintain, regulate or otherwise facilitate expression of the isolated nucleic acid of the invention, such as by way of expression and/or processing of RNA and encoded protein.

Such regulatory nucleotide sequences include an enhancer, promoter, splice donor/acceptor signals, terminator and polyadenylation sequences, although without limitation thereto, as are well known in the art and facilitate RNA and protein expression and/or processing. Selectable markers are also useful whether for the purposes of selection of transformed bacteria (such as bla, kanR and tetR) or transformed mammalian cells (such as hygromycin, G418 and puromycin).

Both constitutive and inducible promoters may be useful for expression of proteins according to the invention.

Non-limiting examples of constitutive promoters operable in mammalian cells include cytomegalovirus promoters, adenoviral promoters, lentiviral promoters and hybrid promoters that include regulatory sequences from more than one source promoter.

Non-limiting examples of inducible promoters are IPTG-inducible, alcohol-inducible, heat shock-inducible, metallothionine-inducible, ecdysone-inducible and tetracycline-repressible systems as are well known in the art.

Also contemplated are tissue-specific promoters that facilitate targeted expression of DNA vaccines to thereby enhance processing and presentation of an encoded immunogen.

Typically, the regulatory nucleotide sequences are present in an expression vector adapted for use in (i)-(iii) above.

The expression vector may also comprise other nucleotide sequences that encode a fusion partner to assist purification. By way of example, fusion partners include a polyhistidine tag, maltose binding protein (MBP), Protein A and glutathione S-transferase (GST). It will also be appreciated that nucleotide sequences encoding protease cleavage sites (e.g. factor Xa and thrombin sites) may be present between the isolated protein and fusion partner to allow subsequent removal of the fusion partner.

By way of example, a particular embodiment of a genetic construct useful for recombinant protein expression in bacteria comprises an isolated nucleic acid of the invention fused to E. coli thioredoxin at the N-terminus and a hexahistidine tag at the C-terminus, constructed using the pBAD-thio expression vector from Invitrogen.

### Immunotherapeutic Compositions, Vaccines and Methods of Treatment

The invention provides immunotherapeutic compositions, preferably vaccines, an immunogenic component of which is an isolated protein of the invention as hereinbefore described.

In a particular embodiment, the immunotherapeutic composition comprises an immunogenic extracellular fragment of a TSP-2 protein, alone or together with a TSP-1 protein fragment and/or a 7TM protein fragment.

The invention also provides a method of immunizing against schistosomiasis, or a related disease or condition, including the step of administering the immunotherapeutic composition to an animal.

In one embodiment, the related disease or condition may be fascioliasis associated with liver fluke infection in cattle, sheep and/or humans.

The invention also provides a method of prophylactic or therapeutic treatment of schistosomiasis, or a related disease or condition, including the step of administering the immunotherapeutic composition to an animal.

Preferably the animal is a human.

By “immunotherapeutic composition” is meant a composition that is capable of eliciting an immune response to one or more immunogenic components of the composition, or to an organism from which said one or more immunogenic components are derived.

As used herein, a “vaccine” is an immunotherapeutic composition that elicits a protective immune response.

It will be appreciated that immunotherapeutic compositions and vaccines may be used therapeutically to treat schistosomiasis or may be used prophylactically to prevent or reduce the severity of schistosome infestation, such as by reducing worm burden and/or liver egg burden.

Typically, although not exclusively, an elicited immune response comprises production of antibodies to an immunogenic, extracellular fragment of a schistosome tegument protein selected from the group consisting of a TSP-1; a TSP-2 protein; and a 7TM protein.

Immunotherapeutic compositions of the invention inclusive of protein-, antibody- and nucleic acid-based therapeutics may be administered in any of a number of forms and routes of administration.

It is preferred that immunotherapeutic compositions, such as vaccines of the invention, include an immunologically-acceptable carrier, diluent or excipient, which in some cases may be an adjuvant. However, it will also be appreciated that an immunologically-acceptable carrier, diluent or excipient may be a substance such as water, saline, alcohol, an organic polymer or other immunologically-inert carrier that merely assists vaccine delivery by appropriately suspending and/or solubilizing vaccine components.

As will be understood in the art, an “adjuvant” means a composition comprised of one or more substances that enhances the immunogenicity and efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; granulocyte-macrophage colony-stimulating factor, combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol' EMA; acrylic copolymer emulsions such as Neocryl A640 (e.g. U.S. Pat. No. 5,047,238); vaccinia or animal poxvirus proteins; sub-viral particle adjuvants such as cholera toxin, or mixtures thereof.

Any safe route of administration may be employed for administering an immunotherapeutic composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is particularly appropriate, for example, for administration of immunotherapeutic compositions such as protein-based and DNA-based vaccines.

It will be appreciated by the skilled person that nucleic acid vaccination is becoming an increasingly used mode of vaccination against schistosomiasis. Such nucleic acid vaccination strategies may include co-administration of other immunologically active molecules such as IL-12 (Zhu et al., 2004, Vaccine 23 78), IL-2 and/or granulocyte-macrophage colony-stimulating factor (Siddiqui et al., 2003, Vaccine 21 2882) and other immunogens such as Sj23 (Zhu et al., 2004, supra), calpain large subunit (Siddiqui et al., 2003, supra) and/or antioxidant molecules such as superoxide dismutase (Cook et al., 2004, Infect. Immun. 72 6112).

In a less preferred embodiment, the invention provides passive immunization by way of administration of an immunotherapeutic composition comprising one or more antibodies which bind an immunogenic, extracellular fragment of a schistosome tegument protein selected from the group consisting of a TSP-1 protein; a TSP-2 protein; and a 7TM protein.

The immunotherapeutic compositions and methods of the invention are preferably practised on humans.

However, veterinary applications are also contemplated. For example, in Asian schistosomiasis S. japonicum uses water buffaloes as a major reservoir and source for transmission of schistosomiasis, particularly in China. It is therefore proposed that the incidence of schistosomiasis could be at least reduced within the human population by treating buffaloes.

### Antibodies

The invention also provides an antibody that binds an immunogenic, extracellular domain fragment of a schistosome tegument protein selected from the group consisting of TSP-1 protein; TSP-2 protein; and 7TM protein.

In one embodiment, the antibody may be produced in a human in response to immunization with an immunogenic fragment of the invention.

In another embodiment, antibodies may be raised against an immunogenic fragment of the invention by immunization of a host animal.

Host animals may be experimental animals such as rabbits, rats, mice and the like.

Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988.

In particular, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the polypeptides, fragments, variants or derivatives of the invention.

The invention also includes within its scope antibodies which comprise Fc or Fab fragments of the polyclonal or monoclonal antibodies referred to above. Alternatively, the antibodies may comprise single chain Fv antibodies (scFvs) against the immunogenic fragments of the invention. Such scFvs may be prepared, for example, in accordance with the methods described respectively in U.S. Pat. No. 5,091,513, European Patent No 239,400 or the article by Winter & Milstein, 1991, Nature 349 293, which are incorporated herein by reference.

Labels may be associated with an antibody of the invention, or antibody fragment, and may be selected from a group including a chromogen, a catalyst, an enzyme, a fluorophore, a chemiluminescent molecule, a lanthanide ion such as Europium (Eu³⁴), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes useful as labels is disclosed in United States Patent Specifications U.S. Pat. No. 4,366,241, U.S. Pat. No. 4,843,000, and U.S. Pat. No. 4,849,338, all of which are herein incorporated by reference. Enzyme labels useful in the present invention include, for example, alkaline phosphatase, horseradish peroxidase, luciferase, β-galactosidase, glucose oxidase, lysozyme, malate dehydrogenase and the like. The enzyme label may be used alone or in combination with a second enzyme in solution.

The fluorophore may, for example, be fluorescein isothiocyanate (FITC), tetramethylrhodamine isothiocyanate (TRITL), allophycocyanin (APC), Texas Red, Cy5, Cy3, or R-Phycoerythrin (RPE) as are well known in the art.

### Diagnostic Methods and Kits

It will also be appreciated that the invention provides a method of determining whether an animal has been exposed to, or harbours, a schistosome, said method including the step of determining whether a biological sample obtained from said animal comprises one or more antibodies which bind an immunogenic, extracellular fragment of a schistosome tegument protein selected from the group consisting of a TSP-1 protein; a TSP-2 protein; and a 7TM protein, wherein a presence of said one or more antibodies indicating that said animal has been exposed to, or harbours, a schistosome.

Preferably, the biological sample is serum.

Preferably, the animal is a human.

Reagents for use in diagnosis of schistosomiasis may advantageously be provided in kit form. Such reagents may optionally include positive and negative control antibodies, secondary antibodies, detection reagents and/or reaction vessels (e.g. microtitre plates, test strips or the like).

The kit may also include instructions for use.

With regard to diagnosis, reference is made to Doenhoff et al., 2004, Trends Parasitol 20 35-39 which provides non-limiting examples of methodology and other considerations applicable to detecting schistosomiasis.

Also provided hereinafter are non-limiting examples of detecting serum antibodies by methods such as ELISA and Western blotting.

So that the invention may be understood in more detail, the skilled person is referred to the following non-limiting examples.

### EXAMPLES

### Materials and Methods

Construction and Expression in E. coli and Western blot Analysis of Sm-TSP-1-EC2, Sm-TSP-2-EC2 and Sm-7TMC

The original cloning and characterization of S. mansoni TSP-1 and TSP-2 are described in Smyth et al., 2003, Infect. Immun. 71 2548.

The full length nucleic acid and protein sequences for each tegument protein described herein can be found using the following details.

GenBank Accession number for the nucleic acid sequences: Sm-TSP-1: AF521093; Sm-TSP-2: AF521091; and Sm7-TM: AY576274.

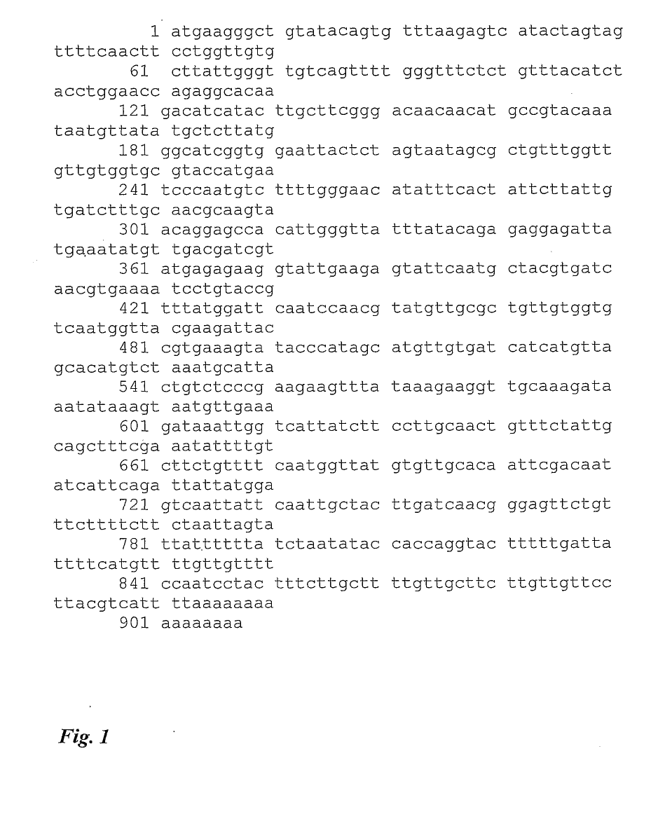

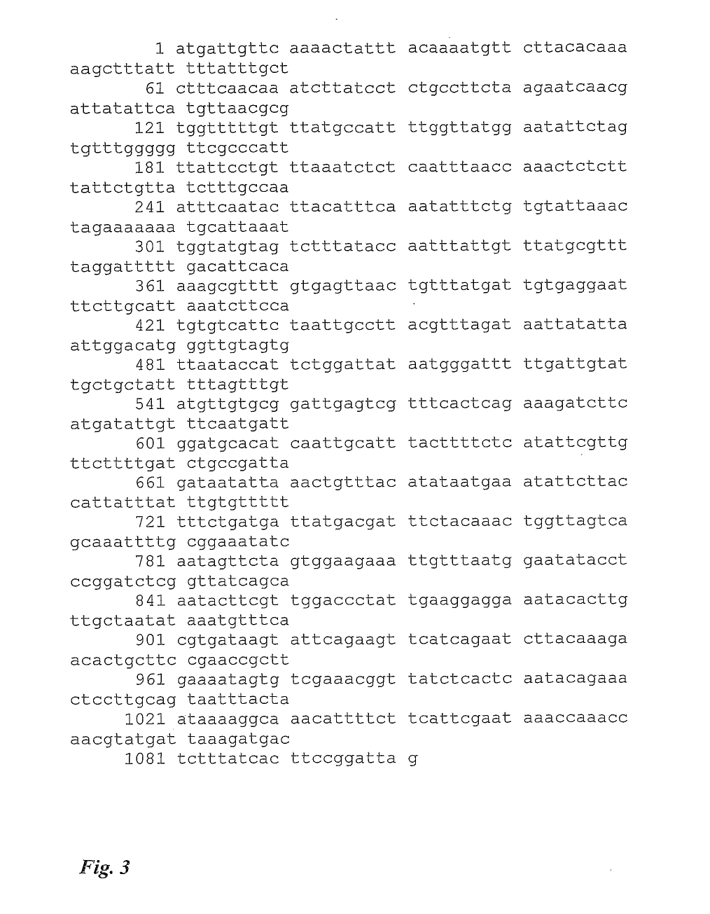

The nucleic acid sequence of Sm-TSP-1 is shown in FIG. 1 and is referred to herein as SEQ ID NO: 1. The nucleic acid sequence of Sin-TSP-2 is shown in FIG. 2 and is referred to herein as SEQ ID NO: 2. The nucleic acid sequence of Sm7-TM is shown in FIG. 3 and is referred to herein as SEQ ID NO: 3.

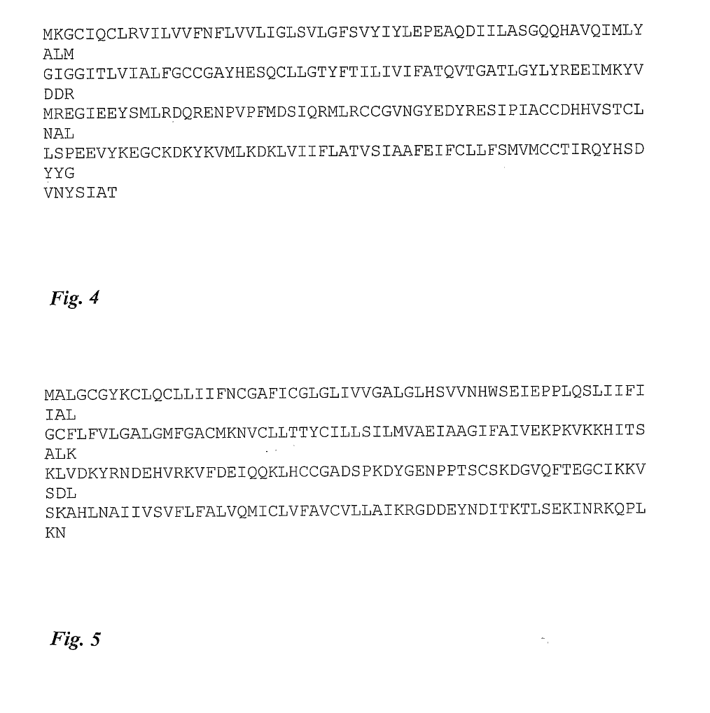

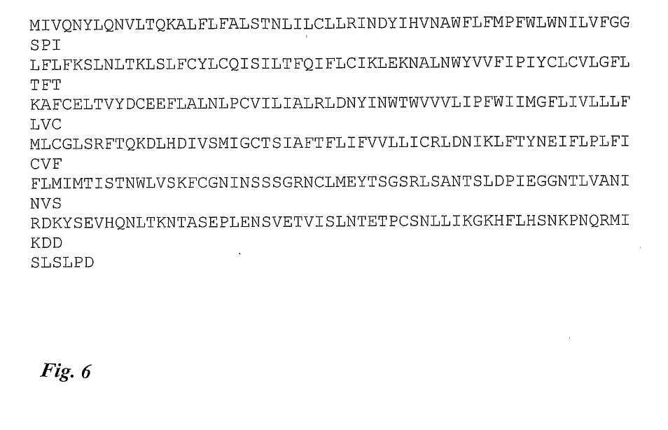

The amino acid sequence of Sm-TSP-1 is shown in FIG. 4 and is referred to herein as SEQ ID NO: 4. The amino acid sequence of Sin-TSP-2 is shown in FIG. 5 and is referred to herein as SEQ ID NO: 4. The amino acid sequence of Sm7-TM is shown in FIG. 6 and is referred to herein as SEQ ID NO: 6.

The respective extracellular domain constructs of each of these are set forth in Table 1.

The nucleotide sequences corresponding to the extracellular domains were respectively cloned using T-ended cloning into the pBAD-thio expression vector (Invitrogen) such that each was fused to the vector-derived Escherichia coli thioredoxin at the N-terminus and V5 and 6His tags at the C-terminus. E. coli TOP-10 cells were transformed with the ligation mix using a heat shock method at 42° C. and cells carrying the recombinant plasmid were selected on LB agar plates for ampicillin resistance. A single colony was selected and grown in 10 ml LB culture broth under ampicillin selection overnight at 37° C. with shaking. The following morning, 1.0 ml of overnight culture was used to seed 100 ml of LB-ampicillin and grown with shaking at 37° C. until an OD₆₀₀ of 0.5 was reached. Protein expression was then induced by adding 20% arabinose to a final concentration of 0.2%. Cultures were grown for another 4 hours before harvesting. To solubilise recombinant protein, cells were lysed with lysis buffer under native conditions as described in the pBAD-thio instruction manual (Invitrogen). Recombinant fusion protein was purified using immobilised metal ion-affinity chromatography (Talon) and assessed for purity by SDS-PAGE analysis and Western blotting with commercially available antibodies to both the V5 and 6His epitopes.

A glycerol stock of an E. coli TOP10 clone containing (A) the Sm-TSP-1-EC2/pBAD/Thio-TOPO plasmid, (B) the Sm-TSP-2-EC2/pBAD/Thio-TOPO or (C) Sm-7TMC/pBAD/Thio-TOPO plasmid was made and a stab from the glycerol stock was inoculated into 10 ml of LB (100 μg/ml ampicillin) and shaken overnight at 220 rpm and 37° C., The entire culture was then added to 1 L of LB containing 100 μg/ml ampicillin and shaken at 220 rpm and 37° C. until an OD600 of 0.5 was reached (approximately 3 hours).

Cultures were then induced with 0.2% arabinose for a further 4 hours under the same conditions and subsequently harvested by centrifugation at 4,000 g and 4° C. for 15 minutes. Pellets were stored at −20° C. until lysis.

Pellets were resuspended in a total volume of 80 ml of lysis buffer (50 mM potassium phosphate—pH 7.8, 400 mM NaCl, 100 mM KCl, 10% glycerol, 0.5% Triton X-100, 10 mM imidazole)—or 2.0 ml per 25 ml of the original culture volume—and subjected to 3 freeze/thaw cycles alternating between dry ice and 42° C. The lysed pellets were then centrifuged at 12,000 g and 4° C. for 20 minutes. The resultant supernatant was purified by batch binding via the 6× His tag to TALON metal affinity resin (Clontech) according to the manufacturer's instructions. The wash buffer used was the same formulation as the lysis buffer and the protein was eluted by gravity flow through a column in 1.0 ml fractions of elution buffer (lysis buffer with 250 mM imidazole).

Fractions containing the majority of purified protein were desalted with a PD-10 desalting column (Amersham), eluted with PBS and concentrated to approximately 1.0 mg/ml using an Amicon Ultra centrifugal filtration device (Millipore) with a 5 kDa molecular weight cut-off. Protein concentration was estimated using the Bradford Concentration Assay and the protein was divided into suitable aliquots and stored at −20° C.

### Western Blot Analysis

Analysis of Sm-7TMC, Sm-TSP-1-EC2 and Sm-TSP-2-EC2 were performed by Western blot using a monoclonal antibody directed against the 6× His tag of each recombinant fusion protein. One hundred nanograms of recombinant protein was electrophoresed on a 12% SDS-PAGE gel at 200 V for 1.0 hour at RT (room temperature) and then transferred to nitrocellulose membrane at 200 mA for 1.0 hour at RT. After blocking for 1.0 hour at RT in PBST containing 5% non fat skim milk powder (SMP) (PBST/5% SMP), the membrane was washed 3 times (5 minutes each) with PBST and reacted with a C terminal anti-His antibody conjugated to HRP (anti-His-HRP) (Invitrogen)—1:5,000 dilution in PBST/5% SMP—for 1.0 hour at RT. The membrane was then washed a further 3 times (5 minutes each) in PBST and developed with chemiluminescence (ECL Plus—Amersham) before detection.

Vaccination with Sm-7TMC, Sm-TSP-1-EC2 and Sm-TSP-2-EC2

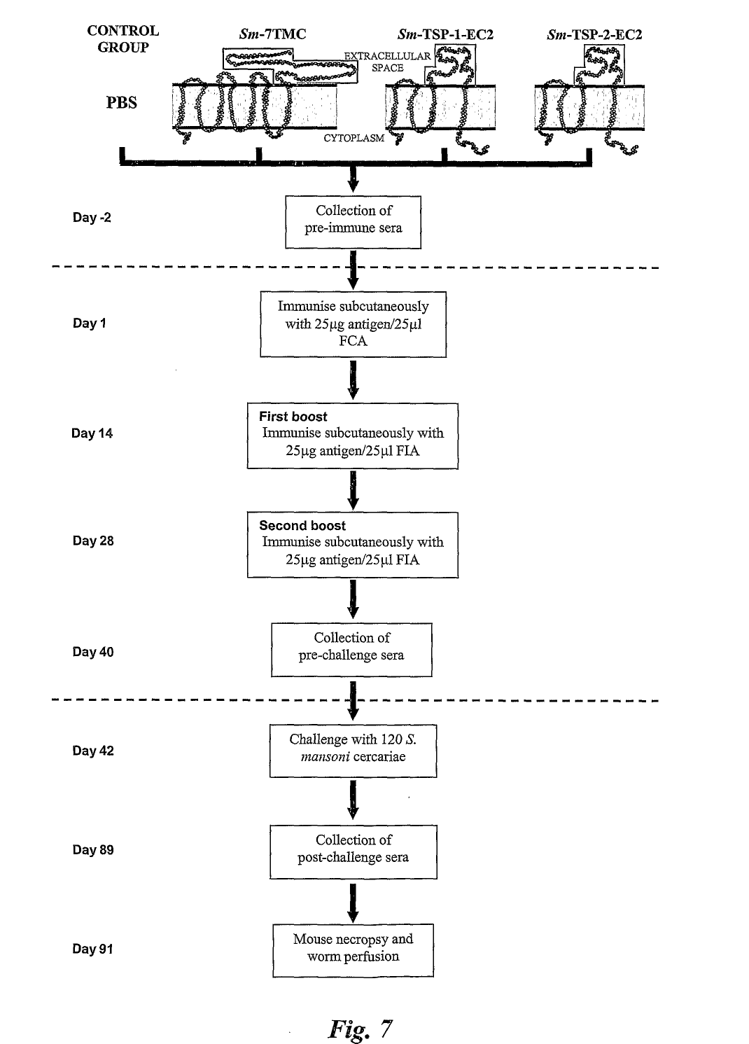

Three groups of 10 female CBA/J mice were each immunised with a separate test antigen (either Sm-7TMC, Sm-TSP-1-EC2 or Sm-TSP-2-EC2) and an additional, control group was immunised with PBS as outlined in FIG. 7. An emulsion containing 25 μg of each recombinant protein (1.0 mg/ml) and an equal volume of Freund's Complete Adjuvant (FCA) was used to subcutaneously immunise each mouse and two subsequent immunisations were similarly administered (with the exception of Freund's Incomplete Adjuvant (FIA) being used instead of FCA) 2 and 4 weeks after the initial immunisation.

Two weeks post-vaccination, mice were challenged with 120 S. mansoni cercariae by abdominal skin penetration (Ham et al., 1984, J. Exp. Med. 159 1371-87). Serum samples were taken at −2, 40 (pre-parasite challenge) and 89 (post-challenge) days after immunisation to assess antibody responses.

### Analysis of Antisera

A pool of serum collected 40 days after the initial immunisation from each test group was analysed for its ability to recognise its corresponding recombinant antigen by Western blot analysis.

The antibody titre of each test pool—as well as an additional pool of serum collected 91 days after the initial immunisation from each test group—was measured by ELISA.

ELISA plates were coated overnight at 4° C. with 500 ng each of recombinant protein—5 μg/ml in coating buffer (0.06 M NaCO₃, pH 9.6)—and then blocked for 1.0 hour at 37° C. with PBST/2% BSA. Following 3 rinses with PBST, serum (diluted 1:100 in PBST) was added to the first well in a row and doubling dilutions were made in each successive well. Plates were then incubated at 37° C. for 1 hour. After 3 rinses with PBST, sheep anti-mouse IgG-HRP (Chemicon)—1:200 dilution in PBST—was added to each well, which were then incubated for 1 hour at 37° C. Following a final 3 washes with PBST, each well was reacted with 50 μl of 2,2′-Azino-Bis (3-Ethylbenziazoline-6-Sulfonic Acid) (ABTS) (Chemicon) and incubated at RT for 20 minutes. Titration endpoints were measured colourimetrically in an automated plate reader (BioRad) at 405 nm.

Pre-immune sera was similarly used as a negative control in both Western blots and ELISAs.

### Detection of Native Protein in Parasite Extracts

Anti-Sm-7TMC serum was used to detect native Sm-7TM in a whole S. mansoni extract. Whole S. mansoni extracts were prepared by incubating approximately 100 worms in 1.0 ml of PBS/1% SDS for 2 hours with rotation at 4° C. The insoluble material was then pelleted by centrifugation and the supernatant aliquoted and stored at −20° C. Ten microlitres of this extract was probed by Western blot analysis with purified anti-Sm-7TMC serum. The membrane was blocked overnight at 4° C. Pre-immune serum was likewise used as a negative control.

Sm-TSP-2-EC2 antiserum was used to precipitate native Sm-TSP-2 from a biotinylated parasite extract as follows. Approximately 100 adult worms were incubated in 1.0 ml of PBS/1% tritonX-100 for 2 hours with rotation at 4° C. The insoluble material was then pelleted by centrifugation and the supernatant transferred to a 1.5 ml centrifuge tube. Polyclonal IgG was then cleared from the extract by binding to 100 μl of protein G sepharose CL-4B (Amersham) for 2 hours with rotation at 4° C. The cleared extract was transferred to a new tube and biotinylated by adding 1.0 mg of EZ-Link NHS-LC Biotin (Pierce) and incubating for 2 hours with rotation at 4° C. Excess biotin was removed by overnight dialysis (at 4° C.) into PBS. Native Sm-TSP-2 was precipitated from this sample by combining 1.0 μl of anti-Sm-TSP-2-EC2 serum with 5.0 μl of extract, 50 μl of protein G sepharose CL-4B and 445 μl of PBST and incubating overnight with rotation at 4° C. The supernatant (unbound fraction) was kept for comparison and protein G sepharose beads were washed several times with PBST and the bound material eluted with 100 μl of 0.1 M glycine, pH 2.8. The eluate was equilibrated by adding 1/10 volume of 1.0 M Tris-HCl, pH 8.0. Samples of the bound and unbound material, 10 μl and 20 μl, respectively, were analysed by Western blot as previously described with the exception that the blocking agent used was 2% bovine serum albumin (BSA) and the antibody conjugate used was streptavidin-HRP. Pre-immune sera was similarly used as a negative control.

### Mouse Necropsy and Estimation of Worm and Liver Egg Burden

Seven weeks after challenge, all mice were euthanased with CO₂ before undergoing necropsy to determine the extent of worm and liver egg burden. Worms were perfused with PBS from the mesenteric veins and the number of male, female and immature parasites were counted. Mouse livers were removed, weighed and digested overnight at 37° C. with 10 ml each of 5% potassium hydroxide. Eggs were collected by centrifugation at 1,000 rpm for 10 minutes and resuspended in 1.0 ml of 4% paraformaldehyde. The amount of eggs in a 5.0 μl aliquot was counted in triplicate, averaged and the number of eggs per mg of liver was calculated. Total worm and liver egg counts were calculated for each group and the reductions in burdens were measured as a percentage compared to the control group. Differences in the means between the control and each test group were assessed statistically using analysis of variance (ANOVA).

### ELISAs Using Experimentally and Naturally Infected Sera

A pool of experimentally infected mouse sera (sampled from the control group used in the challenge experiment) and a panel of sera comprised of 3 samples from humans chronically infected (CI) with S. mansoni and 1 pooled sample of 5 sera from endemic normal (EN) individuals naturally resistant to infection (Correa-Oliveira et al., 2000, Parasitol Today 16 397-9) were tested for their ability to recognise each test antigen by ELISA.

Human sera from a subject with no previous exposure to S. mansoni and the E. coli thioredoxin tag alone were similarly used as negative serum and antigen controls, respectively.

### Screening for Antibodies and Vaccine Studies

### Recombinant Protein Expression

Sm-tsp-1 and Sm-tsp-2 cDNA sequences have been previously described by the inventors (Smyth et al., 2003, Infect. Immun. 71 2548-54) and, as elucidated above, deposited in GenBank with the accession numbers AF521093 and AF521091 respectively. The regions of the cDNAs encoding extracellular loop 2 (Tyr-109—Asp-193 for TSP-1 and Glu-107—His-184 for TSP-2) were amplified by PCR with Pfu polymerase such that they fused in-frame with the N-terminal E. coli thioredoxin and the C-terminal V5 and 6His epitopes encoded by the pBAD/TOPO ThioFusion plasmid (Invitrogen). Ligations of cDNAs into the plasmid and transformations of E. coli TOP10 cells (Invitrogen) with recombinant plasmids were conducted according to the manufacturer's instructions. Subsequent protein expression and solubilization under native conditions were conducted as recommended by the manufacturer.

Recombinant fusion proteins were purified from E. coli lysates under non-denaturing conditions using Triton X-100 in the buffers and cobalt Talon affinity chromatography (BD Biosciences). The identities of the purified proteins were confirmed by Western blotting with antibodies to thioredoxin and the 6His epitopes.

### Immunolocalization

Freshly perfused adult S. mansoni and S. japonicum were fixed in 100% methanol and embedded in Tissue-Tek Optimal Cutting Temperature (OCT) compound (ProSciTech) and cryostatically sectioned into 7.0 μm sections. Sections were then immunolabeled using indirect immunofluorescence as follows. Sections were blocked with 5% skimmed milk powder (SMP) in PBS/0.1% Tween 20 (PBST) and then incubated with mouse anti-TSP-1 or anti-TSP-2 sera (1:25 dilution in SMP) followed by rabbit anti-mouse IgG conjugated to Cy2 (Jackson Immunoresearch: 10 diluted 1:150 in SMP). Sections were counterstained with DAPI (Sigma: 0.1 μg.ml⁻¹ in PBS), which stains nuclei. Slides were mounted with DAKO mounting medium and examined using a confocal microscope (Leica TCS SP2) fitted with Leica DMRE camera. Pre-vaccination serum and serum raised against the thioredoxin tag alone were used as negative controls.

### Assembly of Cohorts

S. mansoni is endemic in the state of Minas Gerais, Brazil. We surveyed villages (córregos) in the Northeastern region of the state, conducting repeated measures prospective surveys as outlined in Table 4. The research team enumerated dwelling structures in each córregos and obtained informed consent from inhabitants using a verbal version of the standard consent form approved by the National Ethics Committee of Brazil.

The research team then monitored water contact of the residents by previously described methods (Bethony et al., 2004 Trop. Med. Int. Health 9 381-9; Bethony et al., 2001 Trop. Med. Int. Health 6 136-45; Gazzinelli et al., 2001 Trop. Med. Int. Health 6 136-45; Kloos et al., 2004 Mem. Inst. Oswaldo Cruz 99 673-81; and Kloos et al., 2006 Act Trop. 97 31-41). Data collection of water contact frequency, fecal exams, and treatment over a 60 month period are shown in Table 4.

Water contact was determined by previously described methods (Bethony et al., 2001, supra; Gazzinelli et al., 2001, supra; and Kloos et al., 2006, supra). In brief, direct observation of water contact (as outlined in Gazzinelli et al., 2001, supra) was first conducted among a subset of the study sample to determine the most frequent water contact activities, duration values of each activity and the body immersion values of each activity (amount of body in water). A survey instrument was constructed and then administered to a subset of the study sample in order to validate the survey (as outlined in Bethony et al., 2001, supra). Water contact frequencies were multiplied by these standardized duration and body immersion values (Bethony et al., 2001, supra; and Kloos et al., 2006, supra).

The clinical forms of schistosomiasis were determined by physical exams performed by experienced physicians from the Centro de Pesquisas Rene Rachou, Belo Horizonte, Brazil. No patient with a clinical form of schistosomiasis (hepatointestinal and hepatosplenic) was included in this study because these forms of schistosomiasis are associated with profound changes in the immune response (reviewed in Pearce et al., 2002 Nat. Rev. Immunol. 2 499-511)). After 60 months of surveillance, we assembled two cohorts of individuals for these studies based upon the criteria in Table 5.

Recombinant Sm-TSP-1 and Sm-TSP-2 were used to screen for specific antibodies in the sera of individuals from Brazil who had been exposed to S. mansoni. The first cohort (n=12) is referred to as putatively resistant (PR) individuals (Correa-Oliveira et al., 1989 Trans. R. Soc. Trop. Med. Hyg. 83 798-804; and Correa-Oliveira et al., 2000 Parasitol. Today 16 397-9) and were defined as (1) negative over the 5 years for S. mansoni infection based on fecal egg count, (2) never been treated with anthelminthic drugs, (3) experienced continuous exposure to infection as evaluated by water contact studies and, (4) have vigorous cellular and humoral responses to crude parasite antigen extracts and/or preparations (Viana et al., 1995 Parasite Immunol. 17 297-304). PR individuals are resistant to infection despite years of exposure to S. mansoni.

The second cohort is referred to as chronically infected (CI) individuals and this group is stratified by the intensity of infection based upon eggs per gram of feces (epg) as detected by the Kato Katz fecal thick smear.

The infection strata are “lightly” infected (1-99 epg; n=20), “moderately” infected (100-399 epg; n=20) and “heavily” infected (>400 epg; n=20). The CI individuals were defined as (1) having been infected with S. mansoni and grouped in one of the 3 infection strata, (2) received anthelminthic treatment until negative (epg=0), (3) had continuous exposure to infection as evaluated by water contact at sites with known transmission, (4) became reinfected with S. mansoni after anthelminthic treatment to levels similar to or higher than baseline infection within 12 months.

Extensive multi-household pedigrees have been constructed in these areas, and to date, no heritable factor has been associated with putative resistance or chronic infection with S. mansoni (Bethony et al., 1999 J. Infect. Dis. 180 1665-73; and Bethony et al., 2002 Am. J. Trop. Med. Hyg. 67 336-43). However, previous research in the area showed a strong heritable (h=42%) factor that associated with intensity of infection (Bethony et al., 2002 supra).

Indirect ELISA with Human Sera.

Serum samples were obtained from whole blood collected into siliconized tubes. Serum was separated by centrifugation at 800 g for 10 min; the resulting serum supernatant was transferred to sterile 1 mL tubes and stored at −80° C. Nunc Maxisorp Surface 96 well plates were coated with 0.5 μg/well of TSP-1 or TSP-2 in 0.06M NaCO₃ (pH 9.6) and stored overnight at 4° C. In the case of assays for IgG2, 96-well plates were first adsorbed overnight at room temperature with 100 μl/well of Poly-Llysine at 1 μg.ml⁻¹ in 50 mM NaCO₃ (pH 9.0). Plates were then washed with PBS and crude antigen was added and incubated in the manner described above. Plates were washed 5 times with PBS and then blocked for 1 h with PBS containing 1% fetal calf serum at RT. Plates were washed 5 times with PBS containing 0.05% Tween 20 (PBST). Serum samples were diluted 1:100 in PBST, and 100 μl/well was added in duplicate to a plate. Plates were incubated overnight at 4° C. and then washed 5 times with PBST as before. One hundred μl of the following dilutions of horseradish peroxidase—conjugated anti-human antibodies (Zymed) were added to each well:1:5,000 of IgG1; 1:1,000 of IgG2, IgG3 and IgG4; 1:800 of IgE. The plates were incubated for 1 h at RT and then washed 10 times with PBST. One hundred μl per well of Ortho-Phenylenediamine (OPD, Sigma) containing 0.03% hydrogen peroxide was then added. Plates were developed for 30 minutes in the dark. The reaction was stopped with 50 μl per well of 30% H₂SO₄ and the Optical Density (OD) was measured at 492 nm on an automated ELISA reader (Molecular Devices). To standardize the assay conditions, the following negative control groups were included on each plate: (1) combined sera of 6 uninfected patients from the United States; (2) combined sera of 6 egg-negative patients without a history of S. mansoni infection from Belo Horizonte, Minas Gerais, Brazil; (3) single serum controls from four egg negative USA volunteers who had not travelled to schistosomiasis endemic areas.

Negative control sera were diluted 1:100 in PBST, and 100 μl/well were added to each plate in duplicate. Assays were repeated if negative sera had a coefficient of variation >10% between plates. Duplicates on a single plate were rejected if they differed by a coefficient of variation of >10% from each other. Assays for a specific isotype were conducted during one run, by use of the same stock of PBS, PBST, PBS with 1% fetal calf serum, and OPD developer to ensure standard conditions across plates. Each plate was tested for correlations (r=0.08) in OD reading by well position. Assay plates in a single run were also tested for correlations (r=0.01) in OD reading by the serial position of the plate during reading.

Vaccination of Mice with Recombinant Proteins

Recombinant TSP-1 and TSP-2 (25 μg per dose in 25 μl) were formulated with an equal volume of Freund's complete (prime) or Freund's incomplete (two boosts) adjuvants. Two groups of 10 female CBA/CaH mice were each immunized with adjuvanted Sm-TSP-1 or Sm-TSP-2. Control groups of 10 mice were immunized with either PBS (trial 1) or 25 μg of recombinant E. coli thioredoxin (trial 2); both PBS and thioredoxin immunogens were formulated with Freund's complete (prime) and incomplete (two boosts) adjuvants. Mice were boosted twice at two-weekly intervals and challenged with 120 S. mansoni cercariae by abdominal skin penetration (Ham et al., 1984 J. Exp. Med. 159 1371-87). Serum samples were taken at −2 (pre-parasite challenge), 40 and 89 (post-challenge) days after immunization to assess antibody responses.

### Mouse Serology

Sera from mice in each vaccine group were screened for recognition of their corresponding recombinant immunogen by ELISA. Microtiter plates (Greiner, Microlon high binding plates) were coated with either TSP-1 or TSP-2 at a final concentration of 3 μg.ml⁻¹ in 0.06 M NaCO₃, pH 9.6. After incubating for 16 h at RT, plates were then blocked with 5% SMP for 2 h at RT. Mouse sera (pooled for trial 1 and individual samples for trial 2) were serially diluted in PBST from 1:100 to 1:6,400,000 and 100 μl was added to each well. Antibodies were allowed to bind for 1 h at RT before addition of HRP-conjugated goat anti-mouse Ig (Silenus) for 1 hr at RT. Three washes with PBST were performed after each incubation step. Peroxidase activity was detected using ABTS substrate (Chemicon) and the optical density was monitored at 405 nm using a Benchmark platereader and Microplate manager (BioRad). Data is reported as antibody end point titers which were defined as the highest dilution of test group sera that yielded an average O.D.+3 standard deviations greater than that obtained in the absence of primary antibody (PBS).

### Mouse Necropsy and Estimation of Worm Burdens and Egg Burdens in Liver and Feces

Seven weeks after challenge, all mice were euthanized with CO₂ and necropsied to determine worm and liver egg burdens. Worms were perfused with PBS from the mesenteric veins and the numbers of male and female adult parasites were counted.

Mouse livers were removed, weighed and digested overnight at 37° C. with 10 ml of 5% potassium hydroxide. Total adult worm burdens and liver egg burdens were calculated for each group and the reductions were measured as a percentage of the parasite burdens in the control group. Fecal egg counts were determined by collecting pooled feces from mice in each group over a 24 hour period, starting on day 48 postinfection.

An equal amount of fecal material (0.5 g) from each group was homogenized by vortexing in PBS and then rotating overnight at 4° C. Each homogenate was pelleted by centrifugation at 500×g for 10 minutes, resuspended in 50 ml of PBS and filtered through a 600 μm mesh followed by a 250 μm mesh. The filtered material was pelleted by centrifugation at 500×g for 10 minutes and resuspended in 10 ml of PBS. The number of eggs in a 100 μl aliquot was counted ten times, averaged and the epg was calculated.

### Statistical Analyses

Statistical analyses of the human antibody responses to recombinant TSP-2 were analysed using a student's t test. For all vaccine trial data, non-parametric Mann-Whitney U-tests were employed because the sample sizes were too small to determine normal distribution. The medians of each individual test group (TSP-1 or TSP-2) were compared with the control group for each analysis. All statistical analyses were conducted using SPSS version 13.

### Results

### Expression of Sm-TSP-1-EC2 and Sm-TSP-2-EC2

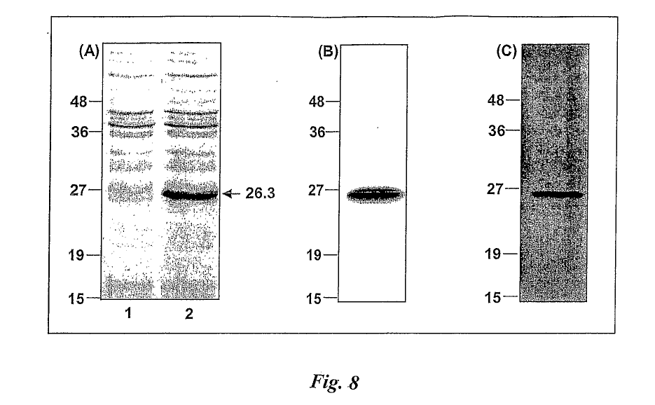

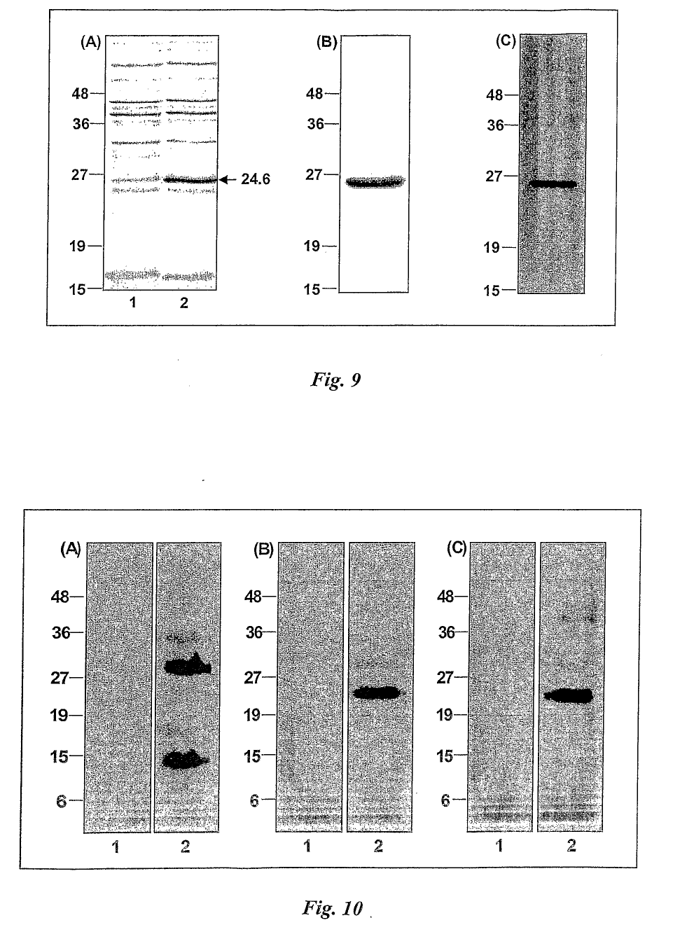

Sm-TSP-1-EC2 and Sm-TSP-2-EC2 were solubly expressed as E. coli thioredoxin fusion proteins. Soluble pre- and post-induction E. coli protein profiles from the Sm-TSP-1-EC2 culture were compared by SDS-PAGE and Sm-TSP-1-EC2 expression was visualised after induction as an unique band with an approximate molecular weight of 26 kDa (FIG. 8A)—the total mass of the fusion protein (thioredoxin and vector tags—16 kDa; and Sm-TSP-1-EC2—10 kDa). Sm-TSP-2-EC2 culture protein profiles were similarly compared and a distinctive post-induction band was observed to migrate at approximately 25 kDa (FIG. 9A)—the molecular weight of Sm-TSP-2-EC2 (9.0 kDa) plus the 16 kDa tag.

The optimal conditions for expression of both antigens were determined to be a 4 hour induction with 0.2% arabinose (data not shown). Scaled-up cultures were induced under these conditions, cells were lysed as previously described and both Sm-TSP-1-EC2 and Sm-TSP-2-EC2 were purified on metal affinity resin using the 6× His tag of the recombinant proteins. Sm-TSP-1-EC2 and Sm-TSP-2-EC2 were assessed for purity and identity by SDS-PAGE (FIG. 8B and 9B, respectively) and Western blot analysis with anti-His-HRP (FIG. 8C and 9C, respectively). The respective protein yields of Sm-TSP-1-EC2 and Sm-TSP-2-EC2 were estimated at 4.0 mg/L and 3.0 mg/L.

### Antibody Response After Immunisation and Challenge

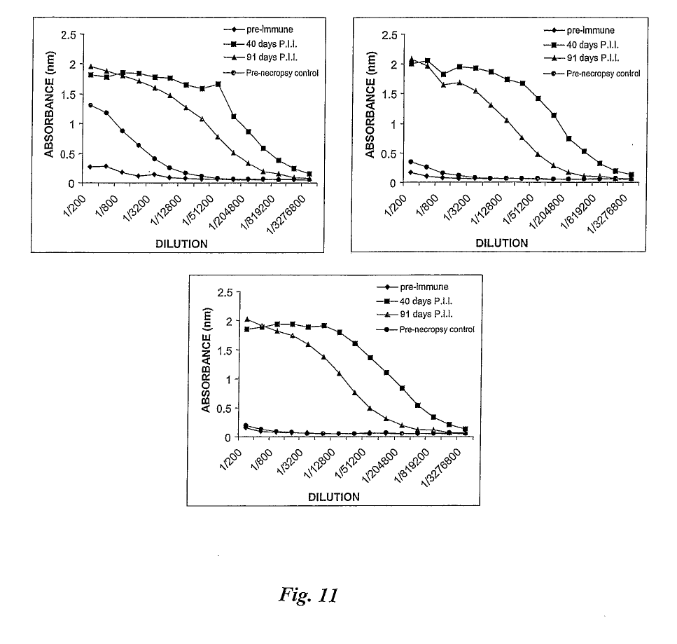

The specificity of each antiserum was tested by probing each purified, recombinant antigen with a pool of corresponding post-vaccination sera (FIG. 10). Western blots containing Sm-TSP-1-EC2 and Sm-TSP-2-EC2 both showed a single band of the size of each recombinant protein and the Western blot containing Sm-7TMC showed two bands corresponding to 29 kDa and 14 kDa—the size of recombinant Sm-7TMC and its degradation product. The degradation product seen in recombinant Sm-7TMC was sequenced by Edman degradation and the resultant sequence (KFRGNINSSS) was confirmed to correspond to residues 4-13 of Sm-7TMC (Lys255 of the complete ORF of Sm-7TM). The three Western blots showed no significant reaction when probed with pre-immune sera. Each of the three antigens generated a strong and specific antibody response; all antisera showed a titre of 1:1,634,800 at 40 days post-initial immunisation but prior to parasite challenge (FIG. 11). A reduction in antibody levels for each group was observed when the antisera were tested at 91 days post-initial immunisation with the anti-Sm-7TMC and anti-Sm-TSP-1-EC2 sera both showing a titre of 1:204,800 and the anti-Sm-TSP-2-EC2 sera exhibiting a titre of 1:409,600.

Additionally, a pool of mouse sera taken from the control group 2 days prior to necropsy (91 days after the start of the trial and 49 days after parasite challenge) was tested for the presence of antibodies against each native antigen by assessing its reactivity to each recombinant protein. Sera from infected mice displayed the highest titre against recombinant Sm-TSP-2-EC2 (1:3,200) (FIG. 11A) whereas only low titres (indeterminate due to the starting dilution used) against Sm-TSP-1 (FIG. 11B) and Sin-7TM (FIG. 11C) were seen. Recombinant thioredoxin alone did not react with these sera (data not shown).

### Native Protein Detection by Antisera

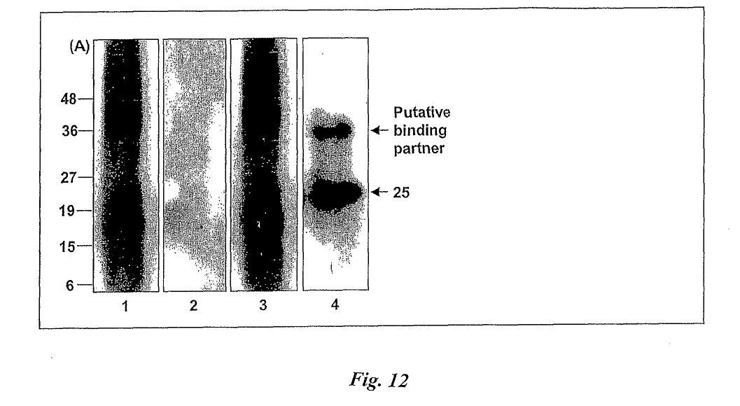

The capacity of each antiserum to recognise its corresponding native antigen was determined by using them to probe solubilized S. mansoni extracts. Native Sm-TSP-2 was immunoprecipitated from a biotinylated, TritonX-100 solubilised worm preparation by anti-Sm-TSP-2-EC2 serum as evidenced by the presence of a 25 kDa band (the approximate predicted molecular weight of full-length Sm-TSP-2) upon SDS-PAGE analysis and streptavidin detection of the precipitated material (FIG. 12A). No significant reaction was seen when pre-immune serum was used in the immunoprecipitation. Interestingly, a 36 kDa protein was also seen in this sample which appeared to have co-precipitated with the immune complex. This band was not evident when the membrane containing the precipitated material was stripped and reprobed by Western blotting with Sm-TSP-2-EC2 serum (data not shown), indicating that the 36 kDa band might correspond to a putative ligand of Sm-TSP-2 and not just an artefact of antibody binding.

Native Sm-7TM was detected by anti-Sm-7TMC serum as demonstrated by reaction with a unique 39 kDa protein (the approximate molecular weight of full-length Sin-7TM) on a Western blot containing an SDS-solubilised S. mansoni extract (FIG. 12B).

The migration of Sm-7TM at this size would seem to indicate that none (or at least not all) of the receptor's five predicted glycosylation sites are being used, as the linkage of any glycans to these residues would increase the molecular weight of Sm-7TM by 2-3 kDa per sugar.

The common reactive bands observed on all of these Western blots are probably mouse immunoglobulin light chain (25 kDa) and various other fragments of IgG that are partially denatured and reduced due to their ability to be detected with just secondary antibody. The presence of mouse immunoglobulin in a schistosome extract is not unusual given the parasite's ability to bind the Fc portion of IgG (Loukas et al., 2001). Further, as SDS was required to solubilise Sm-7TM, the extract was unable to be pre-cleared of IgG using protein G. It should be noted that the presence of SDS precluded the use of native immunodetection methods, such as immunoprecipitation, to detect Sin-7TM. A specific reaction between anti-Sm-TSP-1-EC2 serum and native Sm-TSP-1 could not be detected.

### Worm and Liver Egg Burdens

To determine whether or not vaccination with Sm-7TMC, Sin-TSP-1-EC2 or Sm-TSP-2-EC2 could induce protective immunity against S. mansoni, vaccinated and control mice were each challenged with 120 cercariae and the number of total and adult worms and liver eggs were counted 7 weeks after infection. The results were graphically represented as “reductions in burden” as compared to the control group and are also summarised in Tables 2 and 3.

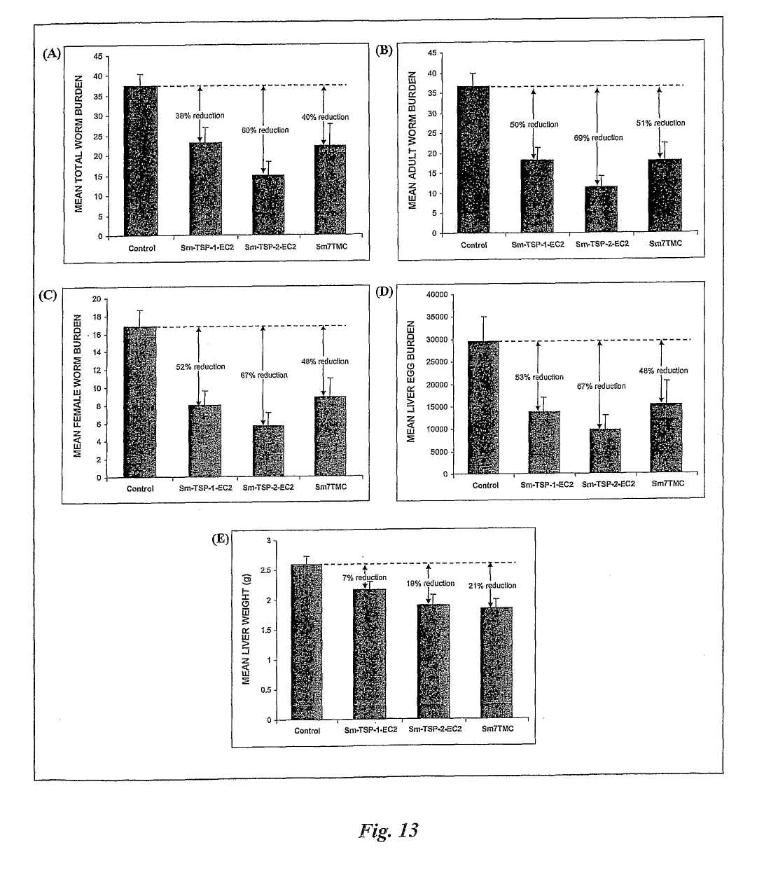

Each vaccinated group showed a statistically significant reduction (P<0.05) in mean total worm burden (including immature worms) of 38-60% (FIG. 13A). There was a 50-69% decrease (P<0.05) in adult worm numbers (FIG. 13B) and a 48-67% reduction (P<0.05) in female worm burdens (FIG. 13C). A 67% reduction (P<0.05) in mean liver egg number was observed for the group vaccinated with Sm-TSP-2-EC2 (FIG. 13D) and respective decreases of 48% and 53% (P=0.075) were seen in the groups vaccinated with Sm-7TMC and Sm-TSP-1-EC2. As expected, these decreases could be strongly correlated with female worm burden reductions (R2=0.9997).

Mean liver weight decreases across the vaccinated groups ranged between 7-21% (P<0.005, FIG. 13E). An anti-fecundity effect was not observed with any vaccinated group (further evidenced by the strong female-egg burden correlation) as there was almost no reduction in the mean amount of liver eggs per female compared to the control animals.

### Antigen Recognition by Naturally Infected Human Sera

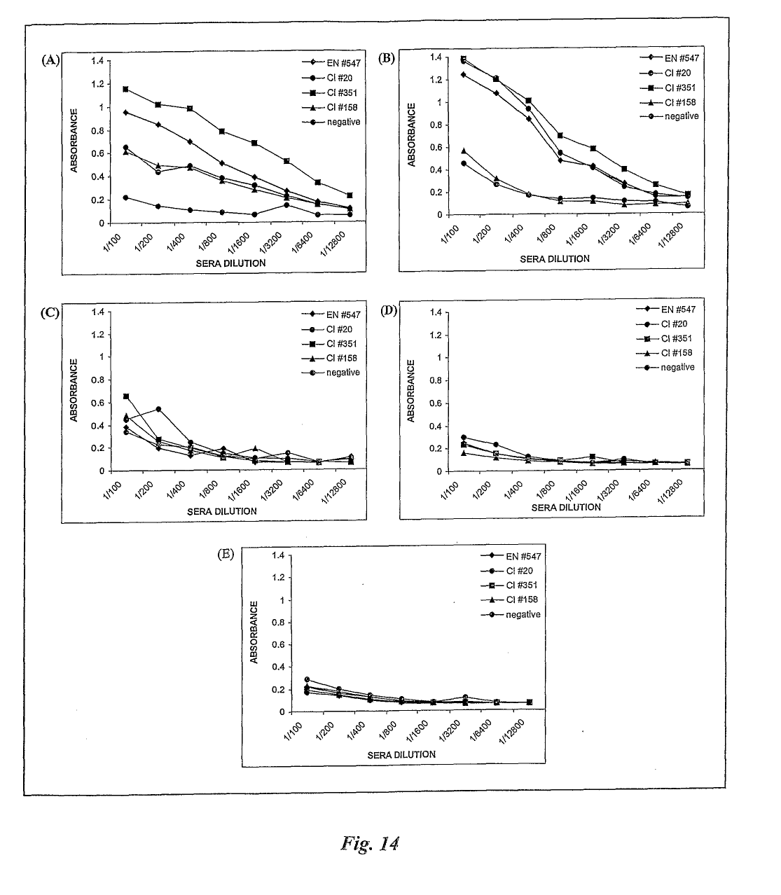

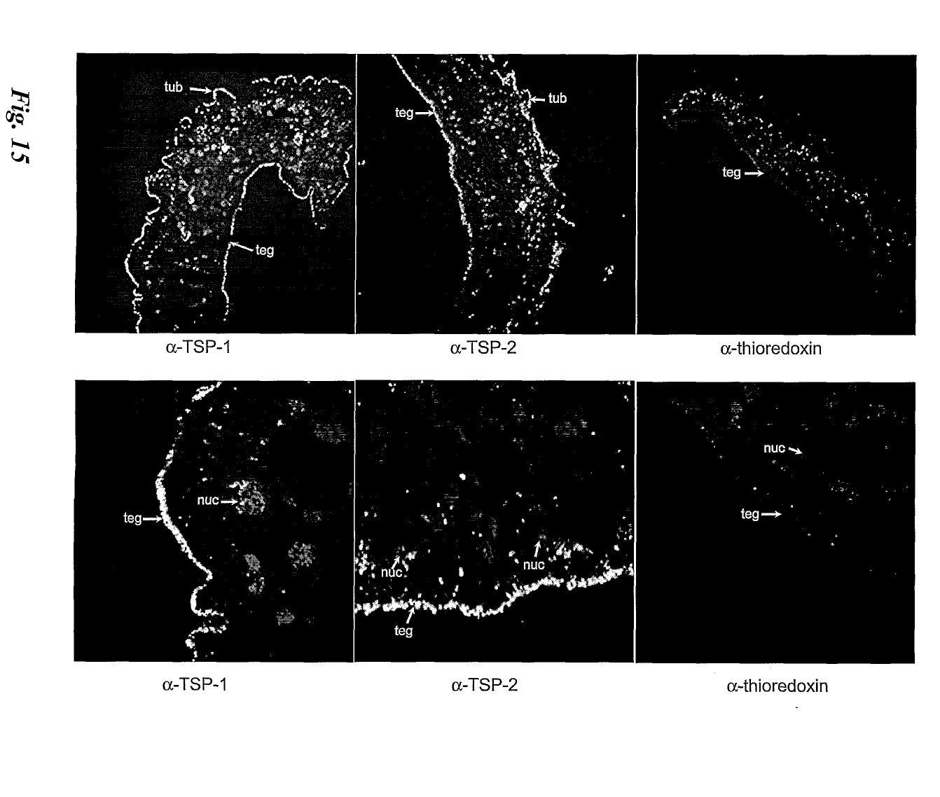

A panel of 4 sera that were positive for antibodies against S. mansoni soluble worm extract—comprised of three samples from CI humans and one pooled sample from 5 EN individuals—and one sample from a subject who had never been exposed to S. mansoni infection were each tested for their ability to recognise TritonX-100-solubilised S. mansoni extract and all three recombinant antigens (FIG. 14).

Titres of antibodies against S. mansoni TritonX-100-soluble worm extract for all naturally infected sera (FIG. 14A) ranged from 1:12,800 (CI serum #351) to 1:1,600 (CI serum #158). The pooled EN serum and CI sera #351 and #20 reacted strongly with Sm-TSP-2-EC2, exhibiting respective titres of 1:1,600, 1:3,200 and 1:800 (FIG. 14B).

Anti-Sm-TSP-1 antibody levels were highest in CI sera from subjects #351 and #20 but were still markedly lower (titres were indeterminate due to the starting dilution used) than those generated in response to Sm-TSP-2. The other two schistosomiasis positive sera did not show anti-Sm-TSP-1 responses (FIG. 14C). CI serum #20 reacted weakly with Sm-7TMC while the other three extract positive sera exhibited no response (FIG. 14D). Recombinant thioredoxin did not react with any of the sera (FIG. 14E).

### Immunolocalization

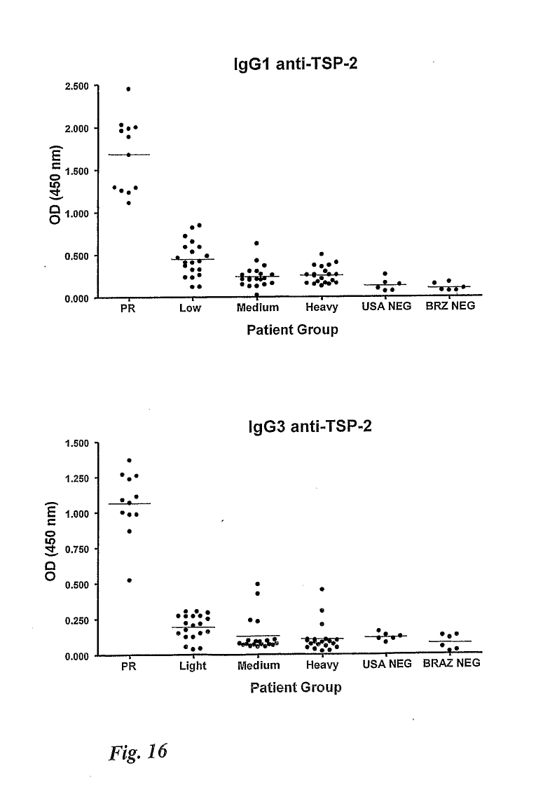

As described above, we expressed and purified the large extracellular loop 2 (major ligand binding domain) of Sm-TSP-1 and Sm-TSP-2 as soluble fusion proteins with E. coli thioredoxin. Mice were vaccinated with the adjuvanted-fusion proteins, and antibodies to both proteins bound exclusively to the tegument of adult S. mansoni; as shown in FIG. 15 weak binding was seen around some of the outermost nuclei that we believe belong to the tegumentary cytons (Jones et al., 2004 supra). Antiserum to the thioredoxin fusion protein did not bind to any schistosome tissues. The protein composition of the S. mansoni tegument was recently reported (van Balkom et al., 2005 J. Proteome Res 4 95-66; and Braschi et al., 2005 Mol. Cell Proteomics 83 798-804) and TSP-2 was one of relatively few integral membrane proteins to be consistently found in the tegument, and not in underlying tissues.

### Screening for Antibodies and Vaccine Studies

Because the TSPs were expressed in the tegument, we used the recombinant proteins to screen for specific antibodies in the sera of individuals from Brazil who were exposed to S. mansoni and were either (1) putatively resistant (PR) (Correa-Oliveira et al., 1989 supra and Correa-Oliveira et al., 2000 supra) or (2) chronically infected (CI) with the parasite. PR individuals are resistant to infection despite years of exposure to S. mansoni. They are defined as: (1) negative over 5 years for S. mansoni infection based on fecal egg counts; (2) never treated with anthelmintic drugs; (3) continually exposed to infection; and (4) maintain a vigorous cellular and humoral immune response to crude schistosome antigen preparations (Correa-Oliveira et al., 1989 supra; Correa-Oliveira et al., 2000 supra; Viana et al., 1995 Parasite Immuno. 17 297-304; and Viana et al., 1994 Trans. R. Soc. Trop Med Hyg 88 466-70).

As shown in FIG. 16 levels of IgG1 and IgG3 against TSP-2 were significantly higher in PR sera than in sera from CI individuals (P<0.001, for both PR and CI). In fact, as also evident from FIG. 16, CI individuals failed to mount any detectable antibody isotype response to TSP-2.

Control sera from unexposed individuals from either the U.S. or Brazil did not mount detectable antibody responses. None of the cohorts tested mounted significant antibody responses to TSP-1 (not shown). CI individuals did however produce moderate to high antibody levels (all IgG subclasses and IgE) against both soluble schistosome egg antigen and soluble adult worm antigen (not shown) (Correa-Oliveira et al., 1989 supra).

Interestingly, the antibody response mounted by the PR individuals against TSP-2 consisted exclusively of the cytophilic antibodies, IgG1 and IgG3 (but not other IgG subclasses nor IgE)—IgG1 and IgG3 are not the antibody isotypes commonly associated with chronic helminth infections (these are IgG4 and IgE) (Hoffmann et al., 2002 Adv. Parasitol. 52 265-307).

Studies in Brazil (Ribeiro de Jesus et al., 2000 Infect. Immun. 68 2797-803) and Egypt (Al-Sherbiny et al., 2003 Act Trop. 88 117-30) assessed the immune responses of resistant and susceptible individuals to a panel of S. mansoni vaccine antigens (mostly those tested by the WHO; Bergquist and Colley 1998 Parasitol. Today 14 99-104), and no single antigen was uniquely recognized by antibodies from resistant but not chronically infected individuals. Putatively resistant individuals did, however, produce greater amounts of IFN-γ to Sm14 (tested as a vaccine by the WHO) than did chronically infected individuals (Brito et al., 2000 Scand. J. Immunol. 51 595-601), but this association has not been reported at the humoral level.

Because the TSPs were expressed on the parasite surface and TSP-2 was selectively recognized by antibodies of naturally immune individuals, we assessed their efficacies as vaccines in the mouse model of S. mansoni infection. Different strains of inbred mice have been used for schistosomiasis vaccine trials—BALB/c and BL/6 mice are considered high responders to the S. mansoni irradiated cercariae vaccine and have fewer worms after challenge infection than do moderate responders such as CBA mice (James et al., 1983 Parasite Immunol 5 567-75 and James et al., 1981 Cell Immunol. 65 75-83), however infected CBA mice display a stronger splenic proliferative response and a lesser suppressor T cell response once infections become patent than do high responder mice (Lewis et al., 1981 Infect Immun 32 260-7).

We chose CBA/CaH mice for our vaccine trials on the assumption that a recombinant antigen that elicits a protective response in this strain would likely yield an even greater protective response in high responder mice. In the vaccine trials, mice were immunized three times with adjuvant-formulated recombinant TSP-1 or recombinant TSP-2, then challenged with S. mansoni cercariae.

Control groups received adjuvanted PBS (trial 1) or thioredoxin (trial 2). Antibody endpoint titers and parasitologic data are provided in Table 6. The antibody responses to both recombinant proteins were dominated by IgG1 and IgG2a, with endpoint titers in excess of 1:1,600,000 for IgG1 and 1:400,000 for IgG2a, against both antigens prior to parasite challenge. By necropsy (91 days after the final vaccination), titers had dropped to 1:800,000 for IgG1 and 1:200,000 for IgG2a, for both antigens. Mice vaccinated with either TSP antigen had significantly lower worm burdens and liver egg burdens than did control mice (Table 6). Vaccination with TSP-2 resulted in a 57% reduction in adult worm burdens and a 64% reduction in liver egg burdens compared to control animals (mean values of both trials combined). Vaccination with TSP-1 resulted in a 34% reduction in mean adult worm burdens and a 52% reduction in mean liver egg burdens. To assess the effects of the vaccines on reducing parasite transmission, fecal egg counts were measured (Trial 2 only), and feces from mice vaccinated with TSP-1 and TSP-2 had 69% and 65% fewer eggs respectively (P<0.0001) than control animals. Vaccination with the

TSPs significantly reduced the numbers of eggs in the liver, the major cause of pathology in schistosomiasis, and in our opinion, a more meaningful endpoint than adult worm burden for an anti-pathology vaccine. Vaccination also resulted in highly significant reductions in fecal egg outputs, highlighting that the TSP vaccines not only reduce parasite load in the host but also in the environment, thereby reducing transmission.

### Discussion

Proteins located on the outer surfaces of parasites are exposed to the host environment and are, therefore, subject to recognition by the host immune response. In this study we have assessed the vaccine efficacies of the major extracellular loops of three tegumental proteins of S. mansoni by their ability to induce a protective immune response in mice after parasite challenge.

Vaccination induced high levels of specific antibodies in each group as confirmed by Western blot analyses and ELISAs of sera taken 40 days post-initial immunisation. Specific antibody responses were still high 91 days after the initial immunisation (49 days post-challenge) even though they were lower than the 40 day responses.

Regarding the 91 day responses to Sm-7TMC and Sm-TSP-1-EC2, it was hypothesised that these lower levels were due to the lingering effects of pre-challenge immunisation, rather than stimulation by natural antigen during parasite challenge, as neither recombinant protein reacted strongly with naturally infected mouse serum.

Contrastingly, the two-fold higher 91 day response to Sm-TSP-2-EC2 might be due to a combined effect of the initial immune response to vaccination with recombinant antigen as well as stimulation of the memory response by native, parasite-derived Sm-TSP-2 during larval challenge, given the strong reactivity of naturally infected mouse serum for Sm-TSP-2-EC2.

The precipitation of native Sm-TSP-2 from a detergent-solubilized parasite, extract by Sm-TSP-2-EC2 antiserum suggests that the recombinant protein contained conformational and/or linear epitopes that mimic those formed by its corresponding natural antigen. Similarly, the reaction between Sm-7TMC antiserum and its native counterpart in an SDS-solubilised parasite extract is indicative that at least the C terminal tail of the recombinant antigen, resembles the C-terminus of native Sm-7TM and may simulate antigen presentation in a natural infection.

The isolation of a possible binding partner for Sm-TSP-2 is an intriguing find, however, it is difficult to speculate on its identity given the unknown function of Sm-TSP-2 and the unknown location of the domain responsible for binding the ligand (due to the antisera precipitating the entire native protein). Tetraspanins are membrane-spanning molecules present on nearly all animal cell types. They mediate processes such as lymphocyte activation, synapse development, cell signalling, adhesion and endocytosis through the recruitment of an array of extracellular and cytoplasmic proteins. The pleiotropic interactions that could be mediated by Sm-TSP-2 further hinders insight into the nature of the co-precipitate; it could be a parasite-derived intracellular signalling molecule or a ligand originating from the host environment that is involved in immunologic masking. Or perhaps it is even another schistosome tegumental protein, given the proclivity of tetraspanins to form homo- and heterodimeric membrane signalling complexes. The attainment of a larger amount of coprecipitated product will permit sequencing of this protein by tandem mass spectrometry, the result of which may elucidate the function of Sm-TSP-2.

The resemblance of Sm-TSP-2-EC2 to the corresponding domain found in native Sm-TSP-2 is further evidenced by the strong reaction between Sm-TSP-2-EC2 and naturally and experimentally infected sera. An interesting observation is the high levels of anti-Sm-TSP-2-EC2 antibodies present in EN sera #351. Longitudinal studies conducted in S. mansoni endemic areas of Brazil over the past 10 years have identified a small (less than 5% of the infected population) group of individuals that are resistant to infection despite years of continual exposure. These “endemic normals” and/or “putatively resistant” individuals exhibit a markedly different immune response compared to CI individuals (Correa-Oliveira et al., 2000, supra) and it has been hypothesized that antigens recognised by sera from this group are capable of eliciting the protective mechanisms needed to effectively combat the parasite. Before any further conclusions as to the nature of the immune response to Sm-TSP-2 can be reached, however, the immunological profile of this antigen will have to be analysed more comprehensively and its reactivity tested against a larger panel of EN and CI sera.

In contrast to Sm-TSP-2-EC2, both Sm-7TMC and Sm-TSP-1-EC2 were not convincingly recognised by naturally and experimentally infected sera. It may be that the extracellular epitopes of Sm-7TM and Sm-TSP-1 (such as those possessed by Sm-7TMC and Sm-TSP-1-EC2) are masked by host-acquired antigen or are being concealed by the parasite in order to subvert the immune response. There is recent evidence suggesting that the schistosome tegument contains invaginated sub-domains which have been shown to house subsets of membrane proteins, including Sm23 (Racoosin et al., 1999, Mol Biochem Parasitol 104 285-97).

It is possible that Sm-7TM and Sin-TSP-1 are contained in these invaginations and are only briefly or intermittently exposed to the immune system, thus being incapable of generating a significant humoral response. It is also possible that these are not particularly antigenic proteins and require formulation with adjuvant and active immunization to generate an antibody response.

Even if the exact nature of immune exposure to each of the three native antigens remains unclear it is evident that all of them are inducing a protective effect, as demonstrated by the significant reductions in adult worm (50-69%) burdens. The decrease in liver egg burden (48-67%) was also strong and, as expected, reflected the significant decrease in female worm numbers and, presumably, liver size. While a fairly balanced reduction between male and female worm burdens was seen for the groups vaccinated with Sm-7TMC and Sm-TSP-1-EC2, the protective response associated with Sm-TSP-2-EC2 appeared to be more gender-targeted, resulting in a 67% decrease in female worm burden. This is suggestive that Sm-TSP-2 may have a role that is more integral to aspects of female biology, a hypothesis that could be supported by the significantly up-regulated transcript level of Sm-TSP-2 in female compared to male S. mansoni worms (Smyth et al., 2003, supra).

It is unlikely that any appreciable protective effect is being induced by the thioredoxin tag of each antigen. Firstly, antibodies raised to the tag do not recognise S. mansoni thioredoxin, as shown by the absence of reactivity with any proteins of 11 kDa (the size of S. mansoni thioredoxin) on Western blots containing parasite extract that are probed with antiserum against the recombinant proteins. Secondly, experimentally infected mouse sera failed to react with recombinant thioredoxin.

Additionally, because thioredoxin is an intracellular protein, it is doubtful that it would be exposed to the host immune system during the course of a natural infection. Repeat vaccine trials of Sin-TSP-1-EC2, Sm-TSP-2-EC2 and Sm-7TMC have commenced with the inclusion of a group vaccinated with thioredoxin alone, the results of which will hopefully show that this molecule does not induce protection against parasite challenge.

A vaccine that induces a reduction in adult worm burden is desirable because, as schistosomes fail to multiply in their definitive snail host, it has the potential to considerably reduce pathology and limit parasite transmission. Further, a decrease in egg output (and, therefore, a reduction and/or suppression in granuloma formation) is advantageous as this could avert the development of chronic disease. The efficacy of Sm-TSP-1-EC2, Sm-TSP-2-EC2 and Sm-7TMC as vaccine candidates has been demonstrated by the achievement of these outcomes. Previous studies have shown that the protective effect of vaccine antigens can be increased by the removal of the fusion tag (Soisson et al., 1992, J. Immunol. 149 3612-20), co-administration with immunostimulatory agents such as granulocyte-macrophage colony-stimulating factor (Siddiqui et al., 2003, supra) and immunisation with multi-epitope or “cocktail” antigens (Argiro et al., 2000, Vaccine 18 2033-2038). The conduction of further immunisation trials using Sm-TSP-1-EC2, Sin-TSP-2-EC2 and Sm-7TMC without their thioredoxin tags, expressed in eukaryotic hosts, combinations of these three antigens or with the addition of immunostimulants and other adjuvants may augment the protective effects described here.

As described above using a signal sequence trap, we cloned two cDNAs encoding tetraspanin integral membrane proteins from S. mansoni—Sm-tsp-1 and Sm-tsp-2. We raised antibodies to recombinant TSP-fusion proteins and showed that both proteins are exposed on the surface of S. mansoni. Recombinant TSP-2, but not TSP-1, is strongly recognized by IgG1 and IgG3 (but not IgE) from naturally resistant individuals but is not recognized by chronically infected or unexposed individuals. Vaccination of mice with the recombinant proteins followed by challenge infection with S. mansoni resulted in significant reductions of 57% and 64% (Sm-TSP-2) and 34% and 52% (Sm-TSP-1) for mean adult worm burdens and liver egg burdens, respectively, over two independent trials. Fecal egg counts were reduced by 65-69% in both test groups. Sm-TSP-2 in particular provided protection levels in excess of the 40% benchmark set by the WHO for progression of schistosome vaccine antigens into clinical trials. When coupled with its selective recognition by naturally resistant people, TSP-2 appears to be an efficacious vaccine antigen against S. mansoni.

Mathematical modelling of S. japonicum transmission dynamics in China showed that an anti-fecundity vaccine for the bovine reservoir host (other schistosome species do not have major reservoir hosts) must have about 75% efficacy to ensure reduction and long-term elimination of the parasite in the human population (Williams et al., 2002 Acta Trop. 82 253-62). Therefore vaccination with either TSP-1 or TSP-2 is within the predicted range to have a major impact on elimination of schistosomiasis mansoni. Indeed, we identified an orthologue of Sm-TSP-2 in the ESTs of the Asian schistosome, S. japonicum, that shared more than 90% sequence identity over the first 116 amino acids (not shown), suggesting that a vaccine based on Sm-TSP-2 might also be effective against S. japonicum.

Neither the protective mechanisms nor the developmental stages targeted by the TSP vaccines are known. Sm-tsp-2 mRNA is highly upregulated in intramammalian stages of the parasite including the lung-stage schistosomulum (not shown) (Smyth et al., 2003 Infect. Immun. 71 2548-54).

We believe that antibodies bind to the surface of the lung-stage parasite as well as the adult parasite in the hepatic portal system, where they opsonize the parasite for further attack by complement and/or antibody-dependent cellular mechanisms. Although their functions are unknown, it is now apparent that a family of tetraspanins is expressed in the schistosome tegument. Sm23, a tetraspanin of unknown function with 31% identity to TSP-2, and one of the independently tested WHO vaccine candidates (Bergquist et al., 1998 supra), was immunolocalized to the tegument of S. mansoni (Ham et al., 1985 J. Immunol. 135 2115-20), although recent analyses of the most abundant proteins in the S. mansoni tegument did not detect Sm23 (van Balkom et al., 2005 supra). A potential function of TSPs in the tegument, however, is immune evasion. One of the most intriguing immuno-evasive mechanisms displayed by schistosomes is the adsorption of host molecules onto the tegument, including MHC (Sher et al., 1978 J. Exp. Med. 148 46-57), to mask the parasite's non-self status. Receptors for some of these adsorbed host products have been identified (Loukas et al., 2001 Infect. Immun. 69 3646-51 and Silva et al., 2004 J. Immunol. 151 7057-66) but the mechanism by which MHC is acquired by the parasite is unknown. Many mammalian tetraspanins complex with MHC or HLA (reviewed in Levy and Shoham 2005 Nat. Rev. Immunol. 5 136-48), and interestingly, Sm-TSP-2 co-immunoprecipitated from schistosome extracts with a protein of the same molecular weight as class II heavy a chain (34 kDa) (not shown).

We suggest that tetraspanins in the tegument of schistosomula and adult worms might be receptors for host ligands including MHC, and vaccination induces antibodies that interfere with the interactions between these tetraspanins and their host ligands, thereby blocking critical immuno-evasive strategies and rendering the parasite surface vulnerable to an effective immune response. The unique recognition of TSP-2 by IgG1 and IgG3 antibodies (but not IgE) from individuals that are exposed but resistant to schistosomiasis, and its vaccine efficacy in mice, emphasizes the potential of this molecule as a safe and effective recombinant vaccine for human schistosomiasis.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All patent and scientific literature, algorithms and computer programs referred to in this specification are incorporated herein be reference in their entirety.

TABLE 1
Schistosoma mansoni antigens tested for vaccine efficacy
in this study. GenBank accession numbers are in parentheses.

| | Size (amino | | |
|---|---|---|---|
| Antigen | acids)* | Derived From | Region Encoded |
| Sm-TSP-1-EC2 | 85 | Sm-TSP-1 | Extracellular loop 2 |
| | | (AAN17278) | (tyr-109-asp-193) |
| Sm-TSP-2-EC2 | 78 | Sm-TSP-2 | Extracellular loop 2 |
| | | (AAN17276) | (glu-107-his-184) |
| Sm7TMC | 114 | Sm-7TM | Extracellular C- |
| | | (AAR84066) | terminus |
| | | | (leu-252-asp-366) |

*size of just extracellular loop, not the entire ORF.

TABLE 2
Parasitologic burdens and liver weights for
mice from vaccinated and control groups.

| Mean | | Sm-TSP-1- | Sm-TSP-2- | |
|---|---|---|---|---|
| Burden | Control | EC2 | EC2 | Sm-7TMC |
| Total worms | 37 ± 3 | 23 ± 4 | 15 ± 4 | 22 ± 5 |
| Adult worms | 37 ± 3 | 18 ± 3 | 11 ± 3 | 18 ± 4 |
| Female worms | 17 ± 2 | 8 ± 2 | 6 ± 1 | 9 ± 2 |
| Liver eggs | 29560 ± 5425 | 13779 ± 3150 | 9726 ± 3252 | 15253 ± 5276 |
| Liver weight | 2.59 ± 0.14 | 2.16 ± 0.14 | 1.89 ± 0.18 | 1.80 ± 0.15 |
| (g) | | | | |

TABLE 3
Percent (%) reductions (and P values) in parasite burdens
and liver weights from mice for each vaccinated group.

| Mean | Sm-TSP-1-EC2 | Sm-TSP-2-EC2 | Sm-7TMC |
|---|---|---|---|
| Burden | % | % | % |
| Total worms | 38 (P = 0.0101) | 60 (P = 0.0002) | 40 (P = 0.0271) |
| Adult worms | 50 (P = 0.0007) | 69 (P = 0.0000) | 51 (P = 0.0032) |
| Female worms | 52 (P = 0.0020) | 67 (P = 0.0000) | 48 (P = 0.0120) |
| Liver eggs | 53 (P = 0.0748) | 67 (P = 0.0073) | 48 (P = 0.0749) |
| Liver weight | 7 (P = 0.0490) | 19 (P = 0.0070) | 21 (P = 0.0020) |
| (g) | | | |

TABLE 4
Identification of cohorts of Chronically Infected (CI) and
Putatively Resistant (PR) individuals in the S. mansoni
endemic area of Siquiera (n = 162) adapted from
Gazzinelli et al., 2001 Trop. Med. Int. Health 6 136-45.

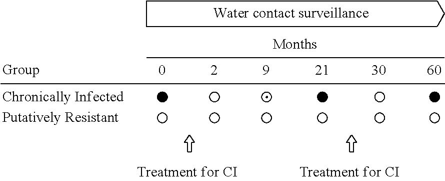

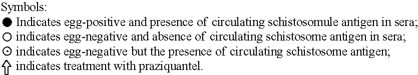

TABLE 5
Inclusion rules for the assembly and maintenance of cohorts

| Resistant group | Rule | |
|---|---|---|
| PR | 1 | Between 15 and 45 years of age. |
| | 3 | Egg-negative at Time 0. |
| | 4 | Egg-negative for 60 months after Time 0. |
| | 5 | Never received anthelmintic treatment (as |
| | | determined by survey). |
| | 6 | Water contact levels at infective sites equal |
| | | to that of chronic patients. |
| | 7 | Strong proliferative response (3 SD above |
| | | control) to STEG*. |
| | 8 | Strong antibody responses (3 SD above control) |
| | | to STEG. |
| | 9 | High production of IFN-γ during in-vitro |
| | | culture with STEG. |

*STEG—S. mansoni Schistosomula TEGumental antigen.
Exclusion rules for the assembly and maintenance of resistant cohorts.
1 Over 45 years of age.
2 Develop clinical schistosomiasis.
3 Egg-positive over 60 month period.
4 Receive anthelmintic treatment outside study regime.
5 Change in water contact levels at infective sites so that different from chronic patients.
6 Change in immune response to crude antigen extracts.
Changes in water contact level and immune responses (proliferative, cytokine or antibody) in individuals was analyzed using Generalized Linear Modeling of Repeated Measures.

TABLE 6
Parasitologic data and antibody titers of mice vaccinated with adjuvanted recombinant Sm-TSP-1, Sm-TSP-2, thioredoxin or PBS, and challenged
with S. mansoni over two independent trials. Statistical analyses were performed using non-parametric Mann-Whitney U-tests on median
values. “n” refers to the number of mice per group (from a total of 10) that survived the trial and were necropsied.

| | | | | Adult worms | | Liver eggs | | Fecal eggs |
|---|---|---|---|---|---|---|---|---|
| | | Adult | Adult worms | Median | Liver eggs | Median | Fecal eggs | Median |
| Adjuvanted | Antibody endpoint | worms - | Mean ± SE | (% reduction) | Mean ± SE | (% reduction) | Mean ± SE | (% reduction) |
| Immunogen | titers | range | (% reduction) | P value | (% reduction) | P values | (% reduction) | P values |

Trial 1

| | | | | | | | | | |
|---|---|---|---|---|---|---|---|---|---|
| Control | Ig | 1:400 | 22-53 | 37.4 ± 3 | 37.5 | 29,560 ± 5425 | 26,233 | ND | ND |
| (PBS) | IgG1 | 1:400 | | | | | | | |
| n = 9 | IgG2a | 1:200 | | | | | | | |
| TSP-1 | Ig | 1:3200,000 | 1-48 | 23.2 ± 4 | 22 | 13,779 ± 3150 | 11,393 | ND | ND |
| | IgG1 | 1:1600,000 | | (38%) | (41%) | (53%) | (57%) | | |
| n = 10 | IgG2a | 1:400,000 | | | P = 0.011 | | P = 0.019 | | |
| TSP-2 | Ig | 1:3200,000 | 1-28 | 14.9 ± 4 | 13.0 | 9,726 ± 3252 | 6,534 | ND | ND |
| | IgG1 | 1:1600,000 | | (61%) | (65%) | (67%) | (75%) | | |
| n = 9 | IgG2a | 1:400,000 | | | P = 0.001 | | P = 0.002 | | |

Trial 2

| | | | | | | | | | |
|---|---|---|---|---|---|---|---|---|---|
| Control | Ig | 1:3200,000 | 68-91 | 73.8 ± 3 | 71 | 36,983 ± 3232 | 37,666 | 1700 ± 152 | 1600 |
| (thioredoxin) | IgG1 | 1:1600,000 | | | | | | | |
| n = 8 | IgG2a | 1:400,000 | | | | | | | |
| TSP-1 | Ig | 1:3200,000 | 37-69 | 52.3 ± 4 | 51 | 18,393 ± 3255 | 16,733 | 520 ± 144 | 400 |
| | IgG1 | 1:1600,000 | | (29%) | (28%) | (50%) | (56%) | (69%) | (75%) |
| n = 10 | IgG2a | 1:800,000 | | | P = 0.001 | | P = 0.004 | | P < 0.0001 |
| TSP-2 | Ig | 1:3200,000 | 0-60 | 34.4 ± 7 | 38 | 14,420 ± 3625 | 14,566 | 600 ± 115 | 500 |
| | IgG1 | 1:3200,000 | | (53%) | (46%) | (61%) | (61%) | (65%) | (69%) |
| n = 10 | IgG2a | 1:1600,000 | | | P < 0.0001 | | P = 0.001 | | P < 0.0001 |

1. An isolated protein comprising an immunogenic, extracellular fragment of a schistosome tegument protein selected from the group consisting of a TSP-1 protein; a TSP-2 protein; and a 7TM protein, wherein the isolated protein is not a full-length schistosome tegument protein.

2. The isolated protein of claim 1 consisting essentially of an immunogenic, extracellular fragment of a schistosome tegument protein selected from the group consisting of a TSP-i protein; a TSP-2 protein; and a 7TM protein.

3. The isolated protein of claim 1 or claim 2 consisting of an immunogenic, extraceliular fragment of a schistosome tegument protein selected from the group consisting of a TSP-i protein; a TSP-2 protein; and a 7TM protein.

4. The isolated protein of claim 1, wherein the schistosome tegument protein is a tetraspanin protein.

5. The isolated protein of claim 4, wherein the schistosomal tetraspanin protein is a TSP-i or a TSP-2 protein.

6. The isolated protein of claim 5, wherein the schistosomal tetraspannin protein is a TSP-2 protein.

7. The isolated protein of claim 6, wherein the immunogenic, extracellular fragment is amino acids 107-184 of a S. inansoni TSP-2 amino acid sequence set forth in SEQ ID NO:5.

8. An antibody which binds the isolated protein of claim 1.

9. An immunotherapeutic composition comprising the isolated protein of claim 1 and an immunologically acceptable carrier, diluent or excipient.

10. An immunotherapeutic composition comprising the antibody of claim 8 and an immunologically acceptable carrier, diluent or excipient.

11. An immunotherapeutic composition comprising the isolated protein of claim 1, the antibody of claim 8 and an immunologically acceptable carrier, diluent or excipient.

12. The immunotherapeutic composition of claim 9 wherein the isolated protein comprises an immunogenic extraceliular fragment of a TSP-1 protein, a TSP-2 protein and/or a 7TM protein.

13. The immunotherapeutic composition of claim 12 comprising an immunogenic extracellular fragment of a TSP-2 protein.

14. The immunotherapeutic composition of claim 9 wherein the immunotherapeutic composition is a vaccine.

15. An isolated nucleic acid that encodes the immunogenic protein of claim 1.

16. The isolated nucleic acid of claim 15, that encodes amino acids 107-184 of a S. mansoni TSP-2 amino acid sequence set forth in SEQ ID NO:5.

17. The isolated nucleic acid of claim 16, comprising nucleotides 321 to 554 of SEQ ID NO:2.

18. A genetic construct comprising the isolated nucleic acid of claim 15 operably linked or connected to one or more regulatory nucleotide sequences.

19. A host cell comprising the genetic construct of claim 18.

20. An immunotherapeutic composition comprising the genetic construct of claim 18 and an immunologically acceptable carrier, diluent or excipient.

21. A method of immunizing against schistosomiasis, or a related disease or condition, including the step of administering to an animal an the immunotherapeutic composition selected from the group consisting of an isolated protein comprising an immunogenic, extracellular fragment of a schistosome tegument protein selected from the group consisting of a TSP-1 protein; a TSP-2 protein; and a 7TM protein, wherein the isolated protein is not a full-length schistosome tegument protein, and a nucleic acid coding for said isolated protein.

22. A method of immunizing against schistosomiasis, or a related disease or condition, including the step of administering the genetic construct of claim 18 to an animal.

23. A method of prophylactic or therapeutic treatment of schistosomiasis, or a related disease or condition, including the step of administering to an animal an immunotherapeutic composition comprising the genetic construct of claim 18 and an immunologically acceptable carrier, diluent or excipient.

24. The method of any one of claims 21 wherein the animal is a mammal.

25. The method of claim 24 wherein the mammal is a human.

26. A method of producing an isolated recombinant protein including the step of expressing the isolated nucleic acid of claim 15 in one or more host cells to thereby produce said isolated protein.

27. The method of claim 26 further including the step of purifying the isolated protein from the one or more host cells.

28. A method of determining whether an animal has been exposed to, or harbours, a schisto some, said method including the step of determining whether a biological sample obtained from said animal comprises one or more antibodies which bind an immunogenic, extracellular fragment of a schistosome tegument protein selected from the group consisting of a TSP-1 protein; a TSP-2 protein; and a 7TM protein, wherein a presence of said one or more antibodies indicates that said animal has been exposed to, or harbours, a schistosonie.

29. The method of claim 28 wherein the biological sample is serum.

30. The method of claim 28 wherein the animal is a human.


SEQUENCE LISTING (6 sequences)

SEQ ID NO: 1 - DNA, 908 bp, Schistosoma mansoni
atgaagggctgtatacagtgtttaagagtcatactagtagttttcaacttcctggttgtgcttattgggttgtcagttttgggtttctctgtttacatctacctggaaccagaggcacaagacatcatacttgcttcgggacaacaacatgccgtacaaataatgttatatgctcttatgggcatcggtggaattactctagtaatagcgctgtttggttgttgtggtgcgtaccatgaatcccaatgtcttttgggaacatatttcactattcttattgtgatctttgcaacgcaagtaacaggagccacattgggttatttatacagagaggagattatgaaatatgttgacgatcgtatgagagaaggtattgaagagtattcaatgctacgtgatcaacgtgaaaatcctgtaccgtttatggattcaatccaacgtatgttgcgctgttgtggtgtcaatggttacgaagattaccgtgaaagtatacccatagcatgttgtgatcatcatgttagcacatgtctaaatgcattactgtctcccgaagaagtttataaagaaggttgcaaagataaatataaagtaatgttgaaagataaattggtcattatcttccttgcaactgtttctattgcagctttcgaaatattttgtcttctgttttcaatggttatgtgttgcacaattcgacaatatcattcagattattatggagtcaattattcaattgctacttgatcaacgggagttctgtttcttttcttctaattagtattattttttatctaatataccaccaggtactttttgattattttcatgttttgttgttttccaatcctactttcttgcttttgttgcttcttgttgttccttacgtcattttaaaaaaaaaaaaaaaa

SEQ ID NO: 2 - DNA, 722 bp, Schistosoma mansoni
aaatggctcttggttgtgggtataagtgtttgcagtgtttgctaattattttcaactgtggagcattcatatgtggtctcgggctgattgtggttggtgcacttgggctccattctgttgtaaatcactggagcgaaattgaacccccactacaatctcttattatcttcattattgctctcggatgcttcttatttgttttgggggctttagggatgtttggagcatgcatgaagaatgtttgtttattaacgacgtattgcattcttctatcaattttaatggttgccgaaatagcagcaggaatatttgctatagtagaaaagcccaaggtcaaaaaacacatcactagtgcattaaaaaaattagtagataagtaccgtaatgacgaacatgttcgaaaagtttttgatgaaatccaacaaaaattacattgctgtggtgctgactctcctaaagattatggcgaaaatccaccgacatcatgttcaaaagatggcgtacaatttacagagggatgtattaaaaaggtcagcgatctaagcaaagcgcacctcaatgctatcatagttagcgtgtttctgttcgcattggtccaaatgatttgtctagtatttgcagtatgtgttctattggctataaagcgcggtgacgatgaatacaatgacattacgaaaacgcttagtgaaaaaataaaccgaaaacaaccattaaaaaattaa

SEQ ID NO: 3 - DNA, 1101 bp, Schistosoma mansoni
atgattgttcaaaactatttacaaaatgttcttacacaaaaagctttatttttatttgctctttcaacaaatcttatcctctgccttctaagaatcaacgattatattcatgttaacgcgtggtttttgtttatgccattttggttatggaatattctagtgtttgggggttcgcccattttattcctgtttaaatctctcaatttaaccaaactctctttattctgttatctttgccaaatttcaatacttacatttcaaatatttctgtgtattaaactagaaaaaaatgcattaaattggtatgtagtctttataccaatttattgtttatgcgttttaggatttttgacattcacaaaagcgttttgtgagttaactgtttatgattgtgaggaatttcttgcattaaatcttccatgtgtcattctaattgccttacgtttagataattatattaattggacatgggttgtagtgttaataccattctggattataatgggatttttgattgtattgctgctatttttagtttgtatgttgtgcggattgagtcgtttcactcagaaagatcttcatgatattgtttcaatgattggatgcacatcaattgcatttacttttctcatattcgttgttcttttgatctgccgattagataatattaaactgtttacatataatgaaatattcttaccattatttatttgtgttttttttctgatgattatgacgatttctacaaactggttagtcagcaaattttgcggaaatatcaatagttctagtggaagaaattgtttaatggaatatacctccggatctcggttatcagcaaatacttcgttggaccctattgaaggaggaaatacacttgttgctaatataaatgtttcacgtgataagtattcagaagttcatcagaatcttacaaagaacactgcttccgaaccgcttgaaaatagtgtcgaaacggttatctcactcaatacagaaactccttgcagtaatttactaataaaaggcaaacattttcttcattcgaataaaccaaaccaacgtatgattaaagatgactctttatcacttccggattag

SEQ ID NO: 4 - protein, 247 aa, Schistosoma mansoni
MKGCIQCLRVILVVFNFLVVLIGLSVLGFSVYIYLEPEAQDIILASGQQHAVQIMLYALMGIGGITLVIALFGCCGAYHESQCLLGTYFTILIVIFATQVTGATLGYLYREEIMKYVDDRMREGIEEYSMLRDQRENPVPFMDSIQRMLRCCGVNGYEDYRESIPIACCDHHVSTCLNALLSPEEVYKEGCKDKYKVMLKDKLVIIFLATVSIAAFEIFCLLFSMVMCCTIRQYHSDYYGVNYSIAT

SEQ ID NO: 5 - protein, 239 aa, Schistosoma mansoni
MALGCGYKCLQCLLIIFNCGAFICGLGLIVVGALGLHSVVNHWSEIEPPLQSLIIFIIALGCFLFVLGALGMFGACMKNVCLLTTYCILLSILMVAEIAAGIFAIVEKPKVKKHITSALKKLVDKYRNDEHVRKVFDEIQQKLHCCGADSPKDYGENPPTSCSKDGVQFTEGCIKKVSDLSKAHLNAIIVSVFLFALVQMICLVFAVCVLLAIKRGDDEYNDITKTLSEKINRKQPLKN

SEQ ID NO: 6 - protein, 366 aa, Schistosoma mansoni
MIVQNYLQNVLTQKALFLFALSTNLILCLLRINDYIHVNAWFLFMPFWLWNILVFGGSPILFLFKSLNLTKLSLFCYLCQISILTFQIFLCIKLEKNALNWYVVFIPIYCLCVLGFLTFTKAFCELTVYDCEEFLALNLPCVILIALRLDNYINWTWVVVLIPFWIIMGFLIVLLLFLVCMLCGLSRFTQKDLHDIVSMIGCTSIAFTFLIFVVLLICRLDNIKLFTYNEIFLPLFICVFFLMIMTISTNWLVSKFCGNINSSSGRNCLMEYTSGSRLSANTSLDPIEGGNTLVANINVSRDKYSEVHQNLTKNTASEPLENSVETVISLNTETPCSNLLIKGKHFLHSNKPNQRMIKDDSLSLPD